(19) United States
(12) Patent Application Publication
(10) Pub. No.: US 2022/0257895 A1
(43) Pub. Date: Aug. 18, 2022

(54) EFFICIENT VACUUM PRESSURE SWING ADSORPTION SYSTEMS AND METHODS

(71) Applicant: ResMed Asia Pte. Ltd., Singapore (SG)

(72) Inventor: Rex Dael NAVARRO, Singapore (SG)

(73) Assignee: ResMed Asia Pte. Ltd., Singapore (SG)

(21) Appl. No.: 17/628,988

(22) PCT Filed: Jul. 31, 2020
(86) PCT No.: PCT/SG2020/050444

§ 371 Date: Jan. 21, 2022

Related U.S. Application Data
(60) Provisional application No. 62/880,886, filed on Jul. 31, 2019.

(51) Int. Cl. A61M 16/10; C01B 13/02; A61M 16/20; A61M 16/00; B01D 53/047; B01D 53/053; B01D 53/04

(52) U.S. Cl. CPC A61M 16/101; C01B 13/0259; A61M 16/201; A61M 16/0072; B01D 53/0476; B01D 53/053; B01D 2259/4533; C01B 2210/0014; C01B 2210/0046; B01D 2256/12; B01D 2257/102; B01D 2259/40009; B01D 2259/402; B01D 53/0446

(57) ABSTRACT

Systems and methods for producing oxygen enriched air using vacuum pressure swing adsorption (VPSA) are disclosed. In one implementation, an oxygen concentrator includes a canister system having at least one canister, a pumping system having at least one motor-controlled pump, a set of valves pneumatically coupling the canister system and the pumping system, and a controller. The canister is configured to receive a gas separation adsorbent. The controller is configured to control operation of the pumping system and the set of valves to: selectively pneumatically couple the motor-controlled pump and the canister so as to pressurize the canister and selectively pneumatically couple the motor-controlled pump and the canister so as to evacuate the canister.

### I. CROSS-REFERENCE TO RELATED APPLICATIONS

The present application claims the benefit of the U.S. Provisional Application No. 62/880,886, which was filed on Jul. 31, 2019 and is incorporated herein by reference.

### II. FIELD OF THE TECHNOLOGY

The present technology generally relates to systems and method for producing oxygen enriched air for treating respiratory disorders. In some implementations, vacuum pressure swing adsorption (VPSA) processes are used to produce the oxygen enriched air.

### III. DESCRIPTION OF THE RELATED ART

A. Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive. Rapidly progressive disorders are characterised by muscle impairment that worsens over months and results in death within a few years (e.g. Amyotrophic lateral sclerosis (ALS) and Duchenne muscular dystrophy (DMD) in teenagers). Variable or slowly progressive disorders are characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

B. Respiratory Therapies

Various respiratory therapies, such as Non-invasive ventilation (NIV), Invasive ventilation (IV), and High Flow Therapy (HFT) have been used to treat one or more of the above respiratory disorders.

1. Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

2. Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

3. Supplementary Oxygen

For certain patients, oxygen therapy may be combined with a respiratory pressure therapy or HFT by adding supplementary oxygen to the pressurized flow of air. When oxygen is added to respiratory pressure therapy, this is referred to as RPT with supplementary oxygen. When oxygen is added to HFT, the resulting therapy is referred to as HFT with supplementary oxygen.

C. Respiratory Therapy Systems

These respiratory therapies may be provided by a respiratory therapy system or device. Such systems and devices may also be used to screen, diagnose, or monitor a condition without treating it. A respiratory therapy system may comprise an oxygen source, an air circuit, and a patient interface.

1. Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Oxygen concentrators may implement cyclic processes such as vacuum swing adsorption (VSA), pressure swing adsorption (PSA), or vacuum pressure swing adsorption (VPSA). For example, oxygen concentrators may work based on depressurization (e.g., vacuum operation) and/or pressurization (e.g., compressor operation) in a swing adsorption process (e.g., Vacuum Swing Adsorption, Pressure Swing Adsorption or Vacuum Pressure Swing Adsorption, each of which are referred to herein as a “swing adsorption process”). Pressure swing adsorption may involve using one or more compressors to increase gas pressure inside one or more canisters that contains particles of a gas separation adsorbent. Such a canister when containing a mass of gas separation adsorbent such as a layer of gas separation adsorbent may be referred to as a sieve bed. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The adsorbed molecules may then be desorbed by venting or exhausting the canister. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a feed gas mixture such as air, for example, is passed under pressure through a canister containing a gas separation adsorbent that attracts nitrogen more strongly than it does oxygen, part or all of the nitrogen will be adsorbed by the adsorbent, and the gas coming out of the canister will be enriched in oxygen. When the adsorbent reaches the end of its capacity to adsorb nitrogen, the adsorbed nitrogen may be desorbed by venting the canister. The canister is then ready for another cycle of producing oxygen enriched air. By alternating pressurization of the canisters in a two-canister system, one canister can be separating (or concentrating) oxygen (the “adsorption phase”) while the other canister is being vented (resulting in a near-continuous separation of oxygen from the air). This alternation results in a near-continuous separation of the oxygen from the nitrogen. In this manner, oxygen enriched air can be accumulated, such as in a storage container or other pressurizable vessel or conduit coupled to the canisters, for a variety of uses including providing supplemental oxygen to users.

Vacuum swing adsorption (VSA) provides an alternative gas separation technique. VSA typically draws the gas through the separation process of the canisters using a vacuum such as a compressor configured to create a partial vacuum within the canisters. Vacuum Pressure Swing Adsorption (VPSA) may be understood to be a hybrid system using a combined vacuum and pressurization technique. For example, a VPSA system may pressurize the canisters for the separation process and also apply a partial vacuum for depressurizing the canisters. In conventional VPSA systems, a dedicated compressor typically compresses the canisters and a separate, dedicated evacuator typically evacuates them.

Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched air are condensed. POCs seek to utilize their produced oxygen as efficiently as possible, in order to minimize weight, size, and power consumption. This may be achieved by delivering the oxygen as series of pulses, each pulse or “bolus” timed to coincide with the start of inspiration. This therapy mode is known as pulsed or demand (oxygen) delivery (POD), in contrast with traditional continuous flow delivery more suited to stationary oxygen concentrators. Many conventional VPSA systems are not well suited for POCs. For example, conventional VPSA systems often include multiple compressors, each of which consumes a significant amount of space and power. A need therefore exists for efficient implementations of VPSA systems for POCs.

2. Air Circuit

An air circuit is a conduit or a tube constructed and arranged to allow, in use, a flow of air to travel between two components of a respiratory therapy system such as the oxygen source and the patient interface. In some cases, there may be separate limbs of the air circuit for inhalation and exhalation. In other cases, a single limb air circuit is used for both inhalation and exhalation.

3. Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### IV. SUMMARY OF THE TECHNOLOGY

Example methods and apparatus of the present technology may involve control of an oxygen concentrator, such as a portable oxygen concentrator (POC), to produce oxygen enriched air as part of a therapy for a respiratory disorder. In some implementations, the oxygen concentrator is controlled to produce oxygen enriched air using VPSA. In some such implementations, the oxygen concentrator efficiently uses a single compressor to pressurize and/or evacuate a canister having gas separation adsorbent disposed therein. For example, the oxygen concentrator may include a single two-piston compressor, two canisters each having gas separation adsorbent disposed therein, and a set of valves configured to selectively connect the input or the output of each piston's cylinder to the canisters. During operation, the valves may be controlled to allow two-piston pressurization of one canister followed by single-piston pressurization and evacuation of both canisters to implement portions of a VPSA cycle.

One aspect of the present disclosure relates to an oxygen concentrator for producing oxygen enriched air using vacuum pressure swing adsorption. The oxygen concentrator comprises: a canister system comprising a first canister for receiving a first gas separation adsorbent, wherein the first gas separation adsorbent is configured to separate at least some nitrogen from a stream of ambient air to produce oxygen enriched air; a pumping system comprising a first motor-controlled pump; a set of valves pneumatically coupling the canister system and the pumping system; and a controller comprising one or more processors. The controller is configured to control operation of the pumping system and the set of valves to: selectively pneumatically couple the first motor-controlled pump and the first canister so as to pressurize the first canister; and selectively pneumatically couple the first motor-controlled pump and the first canister so as to evacuate the first canister.

In some implementations, the pumping system further comprises a second motor-controlled pump and the canister system further comprises a second canister for receiving a second gas separation adsorbent, wherein the second gas separation adsorbent is configured to separate at least some nitrogen from a stream of ambient air to produce oxygen enriched air. In some such implementations, the controller is further configured to control operation of the pumping system and the set of valves to: selectively pneumatically couple the second motor-controlled pump and the second canister so as to pressurize the second canister; and selectively pneumatically couple the second motor-controlled pump and the second canister so as to evacuate the second canister.

In some implementations, the controller is further configured to control operation of the pumping system and the set of valves to: pneumatically couple the first motor-controlled pump and the first canister so as to pressurize the first canister while also selectively pneumatically coupling the second motor-controlled pump and the second canister so as to evacuate the second canister; and pneumatically couple the first motor-controlled pump and the first canister so as to evacuate the first canister while also selectively pneumatically coupling the second motor-controlled pump and the second canister so as to pressurize the second canister.

In some implementations, a pressure of the first canister approaches a first sub-ambient pressure as the first canister is evacuated, and a pressure of the second canister approaches a second sub-ambient pressure as the second canister is evacuated. In some implementations, the first and second sub-ambient pressures range from about 500 to 800 millibars.

In some implementations, the controller is further configured to control operation of the pumping system and the set of valves to: selectively pneumatically couple the first motor-controlled pump, the second motor-controlled pump, and the first canister so as to pressurize the first canister; and selectively pneumatically couple the first motor-controlled pump, the second motor-controlled pump, and the second canister so as to pressurize the second canister.

In some implementations, the controller is further configured to control operation of the pumping system and the set of valves to: pneumatically couple the first motor-controlled pump, the second motor-controlled pump, and the first canister so as to pressurize the first canister while also permitting at least a portion of the oxygen enriched air produced by the first canister to purge the second canister; and pneumatically couple the first motor-controlled pump, the second motor-controlled pump, and the second canister so as to pressurize the second canister while also permitting at least a portion of the oxygen enriched air produced by the second canister to purge the first canister.

In some implementations, the controller is further configured to control operation of the pumping system and the set of valves to: pneumatically couple the first motor-controlled pump, the second motor-controlled pump, and the first canister so as to pressurize the first canister while also permitting a stream of nitrogen enriched air to be exhausted from the second canister; and pneumatically couple the first motor-controlled pump, the second motor-controlled pump, and the second canister so as to pressurize the second canister while also permitting a stream of nitrogen enriched air to be exhausted from the first canister.

In some implementations, a pressure of the first canister approaches an ambient pressure as the stream of nitrogen enriched air is permitted to be exhausted from the first canister, and a pressure of the second canister approaches the ambient pressure as the stream of nitrogen enriched air is permitted to be exhausted from the second canister.

In some implementations, the controller is configured to control operation of the first and second motor-controlled pumps with a single motor. In some implementations, the controller is configured to control operation of the first and second motor-controlled pumps with at least two motors. In some implementations, the first motor-controlled pump comprises a first piston, and the second motor-controlled pump comprises a second piston.

In some implementations, the controller is configured to control operation of the pumping system and the set of valves in a periodic pattern so as to produce oxygen enriched air using vacuum pressure swing adsorption.

In some implementations, the set of valves includes at least one valve connecting either the first canister or ambient to an inlet of the first motor-controlled pump. In some implementations, the set of valves comprises a first subset of valves connecting an outlet of the first motor-controlled pump to either the first canister or a second canister. In some implementations, the set of valves comprises a second subset of valves connecting the first subset of valves to the first canister or to ambient. In some implementations, the set of valves comprises a valve selectively connecting the first canister to ambient.

Another aspect of the present disclosure relates to a method for producing oxygen enriched air using vacuum pressure swing adsorption, the method comprising: selectively pneumatically coupling a first motor-controlled pump of a pumping system and a first canister of a canister system through a set of valves so as to pressurize the first canister, wherein the first canister comprises a first gas separation adsorbent configured to separate at least some nitrogen from a stream of ambient air to produce oxygen enriched air; and selectively pneumatically coupling the first motor-controlled pump and the first canister through the set of valves so as to evacuate the first canister.

In some implementations, the method further comprises: selectively pneumatically coupling a second motor-controlled pump of the pumping system and a second canister of the canister system through the set of valves so as to pressurize the second canister, wherein the second canister comprises a second gas separation adsorbent configured to separate at least some nitrogen from a stream of ambient air to produce oxygen enriched air; and selectively pneumatically coupling the second motor-controlled pump and the second canister through the set of valves so as to evacuate the second canister.

In some implementations, pneumatically coupling the first motor-controlled pump and the first canister through the set of valves so as to pressurize the first canister is performed while also pneumatically coupling the second motor-controlled pump and the second canister through the set of valves so as to evacuate the second canister, and pneumatically coupling the first motor-controlled pump and the first canister through the set of valves so as to evacuate the first canister is performed while also pneumatically coupling the second motor-controlled pump and the second canister through the set of valves so as to pressurize the second canister.

In some implementations, a pressure of the first canister approaches a first sub-ambient pressure as the first canister is evacuated, and a pressure of the second canister approaches a second sub-ambient pressure as the second canister is evacuated. In some implementations, the first and second sub-ambient pressures range from about 500 to 800 millibars.

In some implementations, the method further comprises: selectively pneumatically coupling the first motor-controlled pump, the second motor-controlled pump, and the first canister so as to pressurize the first canister; and selectively pneumatically coupling the first motor-controlled pump, the second motor-controlled pump, and the second canister so as to pressurize the second canister.

In some implementations, pneumatically coupling the first motor-controlled pump, the second motor-controlled pump, and the first canister so as to pressurize the first canister is performed while also permitting at least a portion of oxygen enriched air produced by the first canister to purge the second canister, and pneumatically coupling the first motor-controlled pump, the second motor-controlled pump, and the second canister so as to pressurize the second canister is performed while also permitting at least a portion of oxygen enriched air produced by the second canister to purge the first canister.

In some implementations, pneumatically coupling the first motor-controlled pump, the second motor-controlled pump, and the first canister so as to pressurize the first canister is performed while also permitting a stream of nitrogen enriched air to be exhausted from the second canister, and pneumatically coupling the first motor-controlled pump, the second motor-controlled pump, and the second canister so as to pressurize the second canister is performed while also permitting a stream of nitrogen enriched air to be exhausted from the first canister.

In some implementations, a pressure of the first canister approaches an ambient pressure as the stream of nitrogen enriched air is permitted to be exhausted from the first canister, and a pressure of the second canister approaches the ambient pressure as the stream of nitrogen enriched air is permitted to be exhausted from the second canister.

Of course, portions of the aspects may form sub-aspects of the present technology. Also, various ones of the sub-aspects and/or aspects may be combined in various manners and also constitute additional aspects or sub-aspects of the present technology. Other features of the technology will be apparent from consideration of the information contained in the following detailed description, abstract, drawings and claims.

Yet another aspect of the present disclosure relates to an apparatus comprising: means for receiving a first gas separation adsorbent, wherein the first gas separation adsorbent is configured to separate at least some nitrogen from a stream of ambient air to produce oxygen enriched air; means for generating compressed air comprising a first motor-controlled pump; means for pneumatically coupling the means for receiving and the means for generating compressed air; and means for controlling operation of the means for generating compressed air and the means for pneumatically coupling. The means for generating compressed air and the means for pneumatically coupling are controlled by the means for controlling to: selectively pneumatically couple the first motor-controlled pump and the means for receiving so as to pressurize the means for receiving and selectively pneumatically couple the first motor-controlled pump and the means for receiving so as to evacuate the means for receiving.

### V. BRIEF DESCRIPTION OF THE DRAWINGS

Advantages of the present technology will become apparent to those skilled in the art with the benefit of the following detailed description of implementations and upon reference to the accompanying drawings in which:

FIG. 1A depicts an oxygen concentrator in accordance with one form of the present technology.

FIG. 1B is a schematic diagram of the gas separation system of the oxygen concentrator of FIG. 1A.

FIG. 1C is a side view of the main components of the oxygen concentrator of FIG. 1A.

FIG. 1D is a perspective side view of a compression system of the oxygen concentrator of FIG. 1A.

FIG. 1E is a side view of a compression system that includes a heat exchange conduit.

FIG. 1F is a schematic diagram of example outlet components of the oxygen concentrator of FIG. 1A.

FIG. 1G depicts an outlet conduit for the oxygen concentrator of FIG. 1A.

FIG. 1H depicts an alternate outlet conduit for the oxygen concentrator of FIG. 1A.

FIG. 1I is a perspective view of a disassembled canister system for the oxygen concentrator of FIG. 1A.

FIG. 1J is an end view of the canister system of FIG. 1I.

FIG. 1K is an assembled view of the canister system end depicted in FIG. 1J.

FIG. 1L is a view of an opposing end of the canister system of FIG. 1I to that depicted in FIGS. 1J and 1K.

FIG. 1M is an assembled view of the canister system end depicted in FIG. 1L.

FIG. 1N depicts an example control panel for the oxygen concentrator of FIG. 1A.

FIG. 2A is a schematic diagram of the components of an oxygen concentrator in accordance with one form of the present technology.

FIG. 2B is a schematic diagram of the components of an oxygen concentrator in accordance with one form of the present technology.

FIG. 3A is an example of a valve activation switch timing diagram that may be implemented by the oxygen concentrator of FIG. 2A.

FIG. 3B is a graph illustrating examples of canister pressure cycles that may be implemented by the oxygen concentrator of FIG. 2A.

FIG. 4 is a schematic diagram of the components of an oxygen concentrator in accordance with one form of the present technology.

FIG. 5A is an example of a valve activation switch timing diagram that may be implemented by the oxygen concentrator of FIG. 4.

FIG. 5B is a graph illustrating examples of canister pressure cycles that may be implemented by the oxygen concentrator of FIG. 4.

FIG. 6 is a graph comparing examples of canister pressure cycles that may be implemented by oxygen concentrators using PSA and VPSA processes.

FIG. 7A is a graph illustrating an example of a range of operation that may be implemented by an oxygen concentrator using pressure swing adsorption (PSA) processes.

FIG. 7B is a graph illustrating an example of a range of operation that may be implemented by an oxygen concentrator using vacuum pressure swing adsorption (VPSA) processes.

### VI. DETAILED DESCRIPTION OF THE IMPLEMENTATIONS

Embodiments of the present disclosure are described in detail with reference to the drawing figures wherein like reference numerals identify similar or identical elements. It is to be understood that the disclosed implementations are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

A. Examples of Pressure Swing Adsorption Systems and Methods

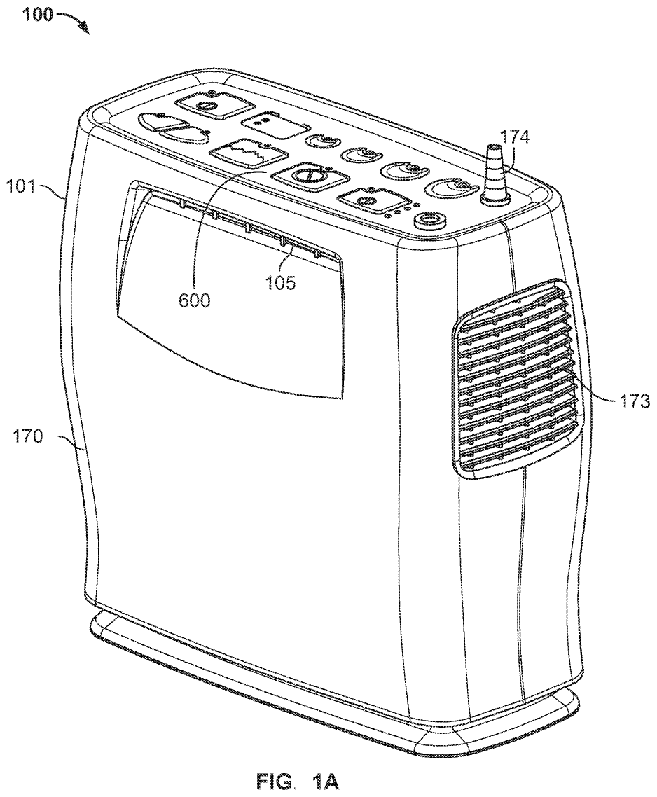

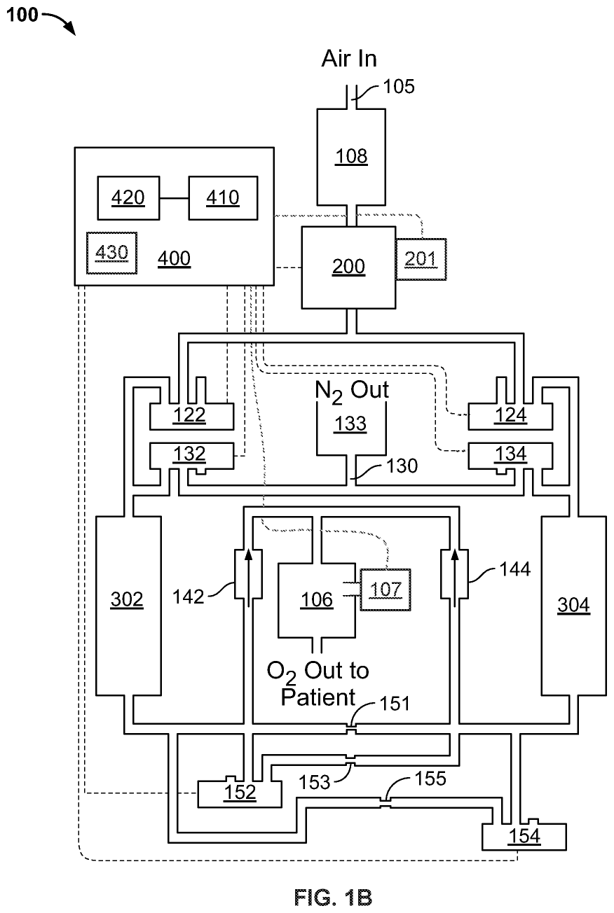

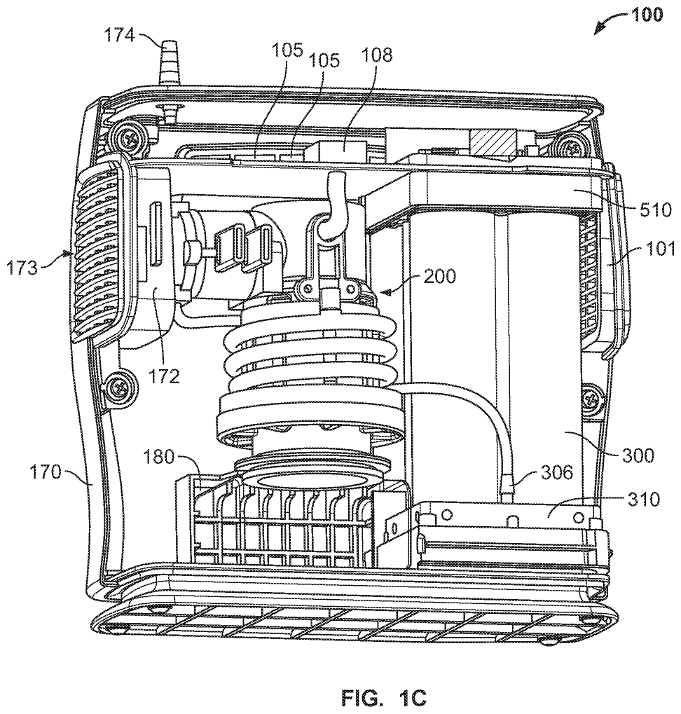

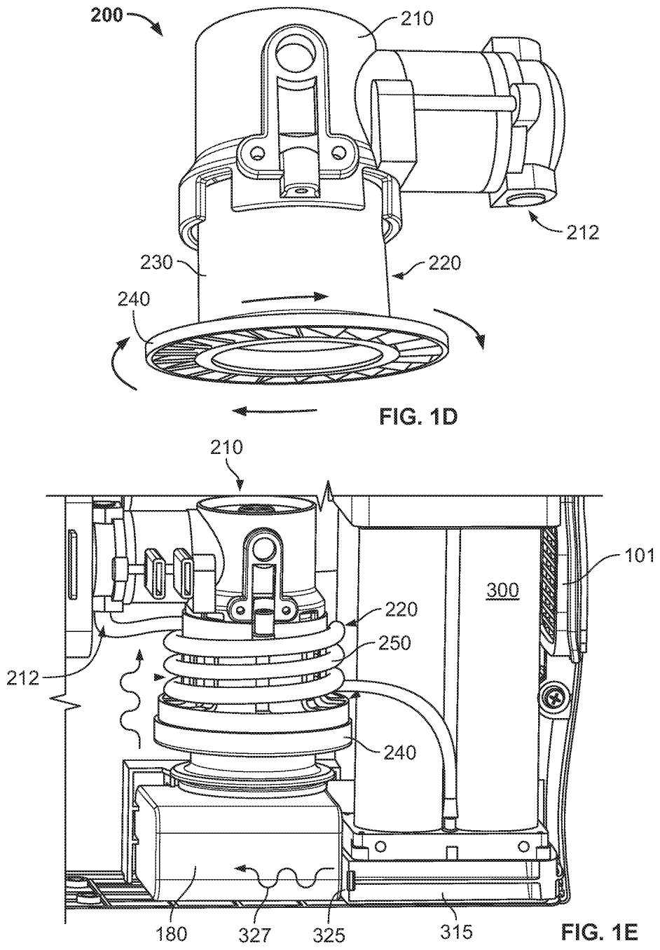

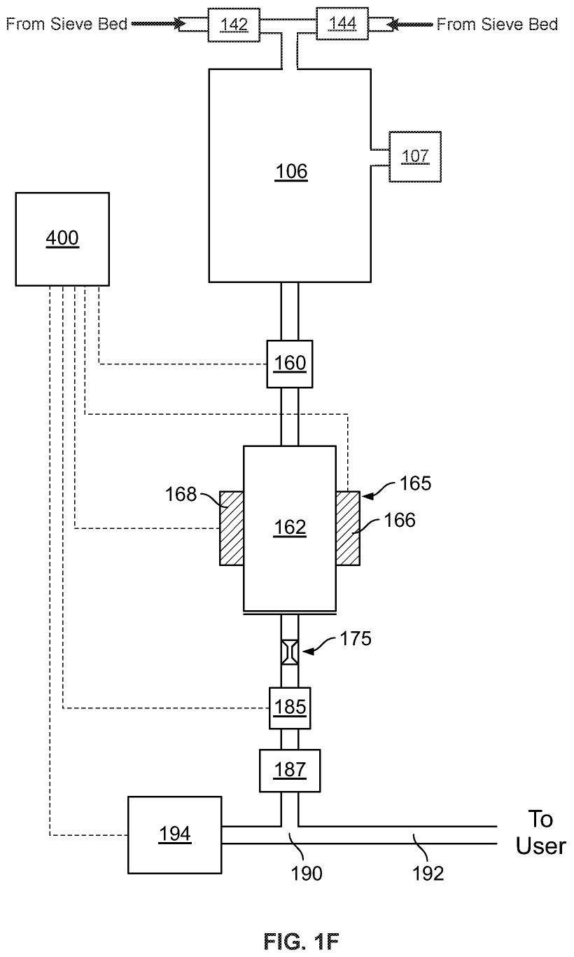

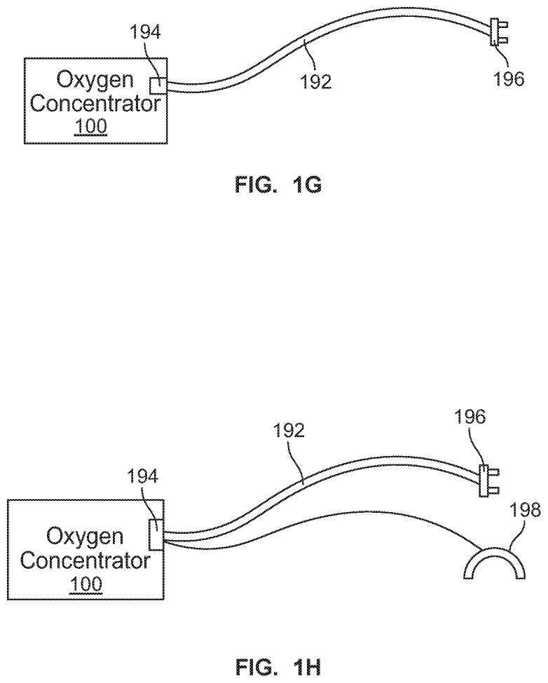

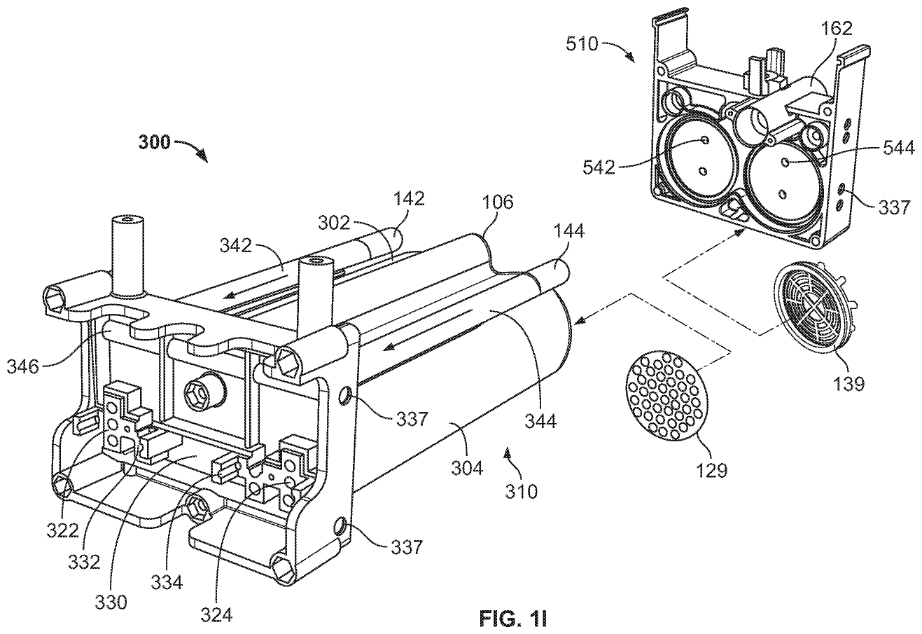

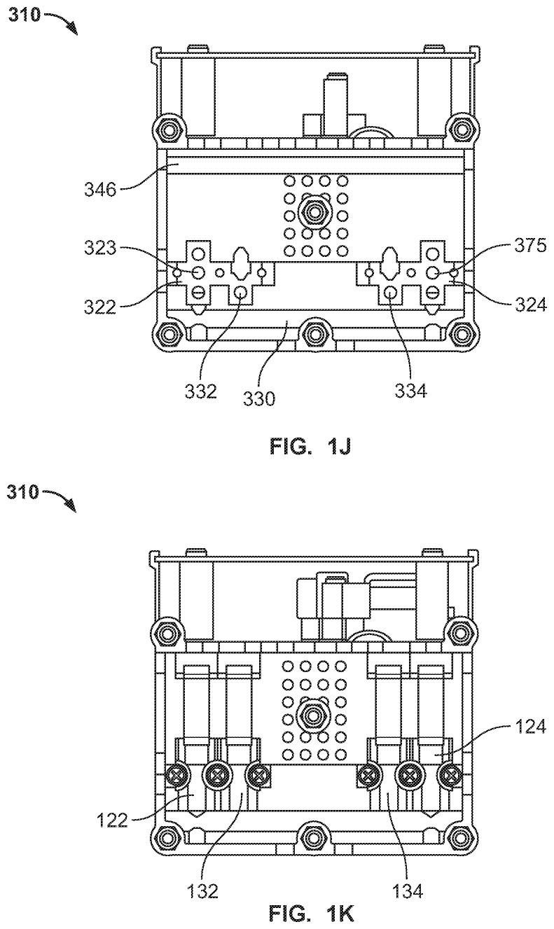

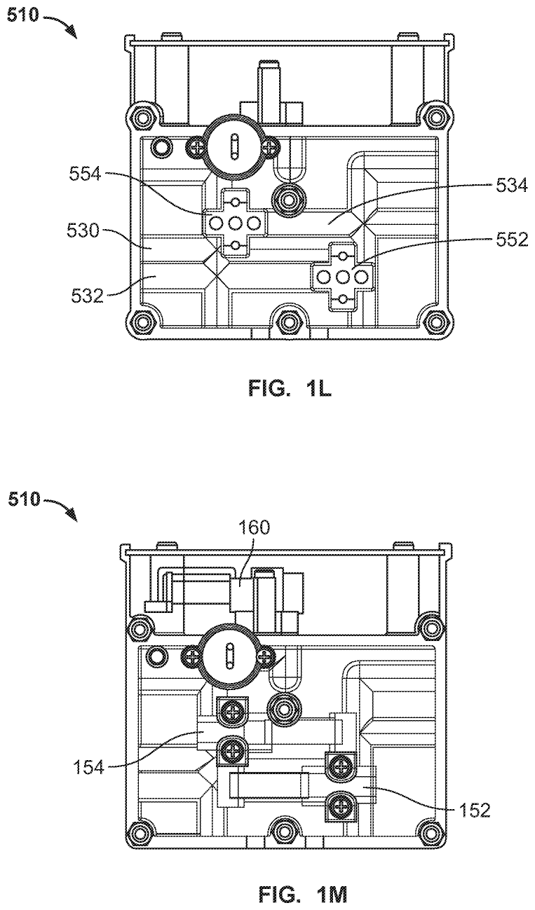

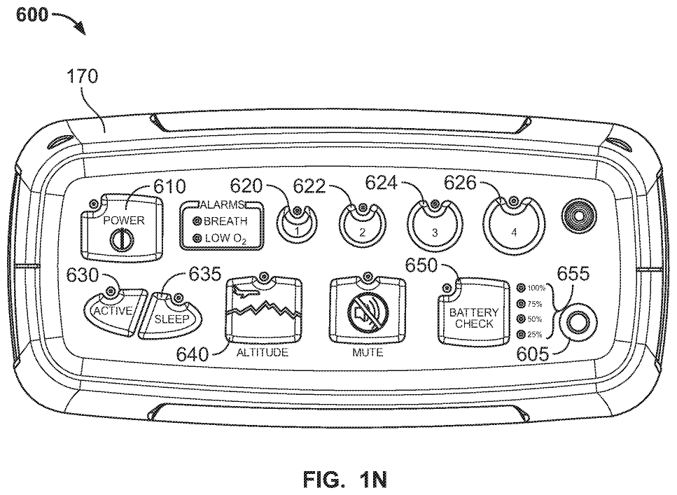

FIGS. 1A-1N illustrate an implementation of an oxygen concentrator 100. As described herein, the oxygen concentrator 100 uses pressure swing adsorption (PSA) processes to produce oxygen enriched air. However, in other implementations, the oxygen concentrator 100 may be modified such that it uses vacuum swing adsorption (VSA) processes or vacuum pressure swing adsorption (VPSA) processes to produce oxygen enriched air.

1. Outer Housing

FIG. 1A depicts an implementation of an outer housing 170 of an oxygen concentrator 100. In some implementations, outer housing 170 may be comprised of a light-weight plastic. Outer housing includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

2. Gas Separation System

FIG. 1B illustrates a schematic diagram of a gas separation system of an oxygen concentrator, such as the oxygen concentrator 100, according to an implementation. The separation system of FIG. 1B may concentrate oxygen within an air stream to provide oxygen enriched air to an outlet system (described below).

Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

Oxygen enriched air may be produced from ambient air by pressurizing ambient air in canisters 302 and 304, which include a gas separation adsorbent. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being drawn into the oxygen concentrator by compression system 200. In an implementation, inlet muffler 108 may be a moisture and sound absorbing muffler. For example, a water absorbent material (such as a polymer water absorbent material or a zeolite material) may be used to both absorb water from the incoming air and to reduce the sound of the air passing into the air inlet 105.

Compression system 200 may include one or more compressors configured to compress air. Pressurized air, produced by compression system 200, may be forced into one or both of the canisters 302 and 304. In some implementations, the ambient air may be pressurized in the canisters to a pressure approximately in a range of 13-20 pounds per square inch gauge pressure (psig). Other pressures may also be used, depending on the type of gas separation adsorbent disposed in the canisters.

Coupled to each canister 302/304 are inlet valves 122/124 and outlet valves 132/134. As shown in FIG. 1B, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release (exhaust) gas from the respective canisters during a venting process. In some implementations, inlet valves 122/124 and outlet valves 132/134 may be silicon plunger solenoid valves. Other types of valves, however, may be used. Plunger valves offer advantages over other kinds of valves by being quiet and having low slippage.

In some implementations, a two-step valve actuation voltage may be used to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat buildup and power consumption to extend run time from the battery. When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. In an implementation, a controller 400 is electrically coupled to valves 122, 124, 132, and 134. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some implementations, the voltages and the durations of the voltages used to open the input and output valves may be controlled by controller 400.

Check valves 142 and 144 are coupled to canisters 302 and 304, respectively. Check valves 142 and 144 are one-way valves that are passively operated by the pressure differentials that occur as the canisters are pressurized and vented. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 to atmosphere while canister 302 is being pressurized. Canister 302 is pressurized until the pressure in canister is sufficient to open check valve 142. Oxygen enriched air produced in canister 302 exits through check valve and, in one implementation, is collected in accumulator 106.

After some time, the gas separation adsorbent will become saturated with nitrogen and will be unable to separate significant amounts of nitrogen from incoming air. This point is usually reached after a predetermined time of oxygen enriched air production. In the implementation described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

During venting of canister 302, outlet valve 132 is opened allowing pressurized gas (e.g., ambient air and/or nitrogen enriched air) to exit the canister to atmosphere through concentrator outlet 130. In an implementation, the vented gases may be directed through muffler 133 to reduce the noise produced by releasing the pressurized gas from the canister. As gas is released from canister 302, the pressure in the canister 302 drops, allowing the nitrogen to become desorbed from the gas separation adsorbent. The released nitrogen enriched air exits the canister through outlet 130, resetting the canister to a state that allows renewed separation of nitrogen from an air stream. Muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air. In some implementations, a canister may be further purged of nitrogen using an oxygen enriched air stream that is introduced into the canister from the other canister.

In an exemplary implementation, a portion of the oxygen enriched air may be transferred from canister 302 to canister 304 when canister 304 is being vented of nitrogen enriched air. Transfer of oxygen enriched air from canister 302 to 304 during venting of canister 304, helps to further purge nitrogen (and other gases) from the canister. In an implementation, oxygen enriched air may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated).

Flow of oxygen enriched air between the canisters is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air, upon pressurization of canister 304, will pass through flow restrictor 151 into canister 302 during venting of canister 302. Additional oxygen enriched air is passed into canister 302, from canister 304, through valve 154 and flow restrictor 155. Valve 152 may remain closed during the transfer process, or may be opened if additional oxygen enriched air is needed. The selection of appropriate flow restrictors 151 and 155, coupled with controlled opening of valve 154 allows a controlled amount of oxygen enriched air to be sent from canister 304 to canister 302. In an implementation, the controlled amount of oxygen enriched air is an amount sufficient to purge canister 302 and minimize the loss of oxygen enriched air through venting valve 132 of canister 302. While this implementation describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the gas flow balance between the two canisters. This may allow for better flow control for venting one of the canisters with oxygen enriched air from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched air flowing from canister 304 toward canister 302 has a flow rate faster through valve 152 than the flow rate of oxygen enriched air flowing from canister 302 toward canister 304 through valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalising the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may be adsorbed by the gas separation adsorbent. Adsorption of water inside the canisters may lead to gradual degradation of the gas separation adsorbents, steadily reducing ability of the gas separation adsorbents to produce oxygen enriched air.

In an implementation, outside air may be inhibited from entering canisters after the oxygen concentrator is shut down by pressurizing both canisters prior to shutdown. By storing the canisters under a positive pressure, the valves may be forced into a hermetically closed position by the internal pressure of the air in the canisters. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shutdown, is at least greater than standard atmospheric pressure (i.e., greater than 760 mmHg (Torr), 1 atm, 101,325 Pa). In an implementation, the pressure in the canisters, at shutdown, is at least about 1.1 times greater than ambient pressure; is at least about 1.5 times greater than ambient pressure; or is at least about 2 times greater than ambient pressure.

In an implementation, pressurization of the canisters may be achieved by directing pressurized air into each canister from the compression system and closing all valves to trap the pressurized air in the canisters. In an exemplary implementation, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate implementation, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 1C, an implementation of an oxygen concentrator 100 is depicted. Oxygen concentrator 100 includes a compression system 200, a canister assembly 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator via outlet 173.

3. Compression System

In some implementations, compression system 200 includes one or more compressors. In another implementation, compression system 200 includes a single compressor, coupled to all of the canisters of canister system 300. Turning to FIGS. 1D and 1E, a compression system 200 is depicted that includes compressor 210 and motor 220. Motor 220 is coupled to compressor 210 and provides an operating force to the compressor to operate the compression mechanism. For example, motor 220 may be a motor providing a rotating component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, as depicted in FIG. 1B, which sends operating signals to the motor to control the operation of the motor. For example, controller 400 may send signals to motor 220 to: turn the motor on, turn motor the off, and set the operating speed of motor.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as a power source for oxygen concentrators due to their long life and light weight. Lithium ion battery packs, however, are dangerous at elevated temperatures and safety controls are employed in oxygen concentrator 100 to shutdown the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply and thus shorten the portable usage time of the oxygen concentrator. In an implementation, a passive cooling system may be used that takes advantage of the mechanical power produced by motor 220. Referring to FIGS. 1D and 1E, compression system 200 includes motor 220 having an external rotating armature 230. Specifically, armature 230 of motor 220 (e.g. a DC motor) is wrapped around the stationary field that is driving the armature. Since motor 220 is a large contributor of heat to the overall system it is helpful to pull heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotating armature 230. In an implementation, air transfer device 240 is coupled to the external armature 230 such that rotation of the external armature causes the air transfer device to create an air flow that passes over at least a portion of the motor. In an implementation, air transfer device includes one or more fan blades coupled to the armature. In an implementation, a plurality of fan blades may be arranged in an annular ring such that the air transfer device acts as an impeller that is rotated by movement of the external rotating armature. As depicted in FIGS. 1D and 1E, air transfer device 240 may be mounted to an outer surface of the external armature 230, in alignment with the motor. The mounting of the air transfer device to the armature allows air flow to be directed toward the main portion of the external rotating armature, providing a cooling effect during use. In an implementation, the air transfer device directs air flow such that a majority of the external rotating armature is in the air flow path.

Further, referring to FIGS. 1D and 1E, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an implementation, the cooled vented gases 327 from canister system 300 are directed toward power supply 180 and toward compression system 200. In an implementation, base 315 of canister system 300 receives the vented gases from the canisters. The vented gases 327 are directed through base 315 toward outlet 325 of the base and toward power supply 180. The vented gases, as noted, are cooled due to decompression of the gases and therefore passively provide cooling to the power supply. When the compression system is operated, the air transfer device will gather the cooled vented gases and direct the gases toward the motor of compression system 200. Fan 172 may also assist in directing the vented gas across compression system 200 and out of the housing 170. In this manner, additional cooling may be obtained without requiring any further power requirements from the battery.

4. Canister System

Oxygen concentrator system 100 may include at least two canisters, each canister including a gas separation adsorbent. The canisters of oxygen concentrator system 100 may be disposed formed from a molded housing. In an implementation, canister system 300 includes two housing components 310 and 510, as depicted in FIG. 1I. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be injection molded or compression molded. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, housing components 310 and 510 may be solvent welded together.

As shown, valve seats 322, 324, 332, and 334 and air pathways 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different x,y,z positions in housing components 310 and 510. In some implementations, a majority or substantially all of the conduits may be integrated into the housing components 310 and 510 to reduce potential leak points.

In some implementations, prior to coupling housing components 310 and 510 together, O-rings may be placed between various points of housing components 310 and 510 to ensure that the housing components are properly sealed. In some implementations, components may be integrated and/or coupled separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plug to seal the passage. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator system 100.

In some implementations, filter 129 may be placed into respective canister receiving portions of housing components 310 and 510 facing the inlet of the respective canisters. The filter 129 removes particles from the feed gas stream entering the canisters.

In some implementations, pressurized air from the compression system 200 may enter air inlet 306. Air inlet 306 is coupled to inlet conduit 330. Air enters housing component 310 through inlet 306 travels through conduit 330, and then to valve seats 322 and 324. FIG. 1J and FIG. 1K depict an end view of housing 310. FIG. 1J depicts an end view of housing 310 prior to fitting valves to housing 310. FIG. 1K depicts an end view of housing 310 with the valves fitted to the housing 310. Valve seats 322 and 324 are configured to receive inlet valves 122 and 124 respectively. Inlet valve 122 is coupled to canister 302 and inlet valve 124 is coupled to canister 304. Housing 310 also includes valve seats 332 and 334 configured to receive outlet valves 132 and 134 respectively. Outlet valve 132 is coupled to canister 302 and outlet valve 134 is coupled to canister 304. Inlet valves 122/124 are used to control the passage of air from conduit 330 to the respective canisters.

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. During pressurization of canister 302, outlet valve 132 is closed and outlet valve 134 is opened. Similar to the inlet valves, outlet valves 132 and 134 are operated out of phase with each other. Valve seat 322 includes an opening 323 that passes through housing 310 into canister 302. Similarly valve seat 324 includes an opening 375 that passes through housing 310 into canister 302. Air from conduit 330 passes through openings 323 or 375 if the respective valves 322 and 324 are open, and enters a canister.

Check valves 142 and 144 (See FIG. 1I) are coupled to canisters 302 and 304, respectively. Check valves 142 and 144 are one-way valves that are passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing 310.

Oxygen enriched air from either canister travels through conduit 342 or 344 and enters conduit 346 formed in housing 310. Conduit 346 includes openings that couple the conduit to conduit 342, conduit 344 and accumulator 106. Thus, oxygen enriched air, produced in canister 302 or 304, travels to conduit 346 and passes into accumulator 106.

After some time, the gas separation adsorbent will become saturated with nitrogen and will be unable to separate significant amounts of nitrogen from incoming air. When the gas separation adsorbent in a canister reaches this saturation point, the inflow of compressed air is stopped and the canister is vented to remove nitrogen enriched air. Canister 302 is vented by closing inlet valve 122 and opening outlet valve 132. Outlet valve 132 releases the vented gas from canister 302 into the volume defined by the end of housing 310. Foam material may cover the end of housing 310 to reduce the sound made by release of gases from the canisters. Similarly, canister 304 is vented by closing inlet valve 124 and opening outlet valve 134. Outlet valve 134 releases the vented gas from canister 304 into the volume defined by the end of housing 310.

While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

In an exemplary implementation, a portion of the oxygen enriched air may be transferred from canister 302 to canister 304 when canister 304 is being vented of nitrogen enriched air. Transfer of oxygen enriched air from canister 302 to canister 304, during venting of canister 304, helps to further purge nitrogen (and other gases) from the canister. Flow of oxygen enriched air between the canisters is controlled using flow restrictors and valves, as depicted in FIG. 1B. Three conduits are formed in housing component 510 for use in transferring oxygen enriched air between canisters. As shown in FIG. 1L, conduit 530 couples canister 302 to canister 304. Flow restrictor 151 (not shown) is disposed in conduit 530, between canister 302 and canister 304 to restrict flow of oxygen enriched air during use. Conduit 532 also couples canister 302 to 304. Conduit 532 is coupled to valve seat 552 which receives valve 152, as shown in FIG. 1M. Flow restrictor 153 (not shown) is disposed in conduit 532, between canister 302 and 304. Conduit 534 also couples canister 302 to 304. Conduit 534 is coupled to valve seat 554 which receives valve 154, as shown in FIG. 1M. Flow restrictor 155 (not shown) is disposed in conduit 534, between canister 302 and 304. The pair of equalization/vent valves 152/154 work with flow restrictors 153 and 155 to optimize the air flow balance between the two canisters.

Oxygen enriched air in accumulator 106 passes through supply valve 160 into expansion chamber 162 which is formed in housing component 510. An opening (not shown) in housing component 510 couples accumulator 106 to supply valve 160. In an implementation, expansion chamber 162 may include one or more devices configured to estimate an oxygen concentration of gas passing through the chamber.

5. Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 1B. The oxygen enriched air leaving the canisters may be collected in an oxygen accumulator 106 prior to being provided to a user. In some implementations, a tube may be coupled to the accumulator 106 to provide the oxygen enriched air to the user. Oxygen enriched air may be provided to the user through an airway delivery device that transfers the oxygen enriched air to the user's mouth and/or nose. In an implementation, an outlet may include a tube that directs the oxygen toward a user's nose and/or mouth that may not be directly coupled to the user's nose.

Turning to FIG. 1F, a schematic diagram of an implementation of an outlet system for an oxygen concentrator is shown. A supply valve 160 may be coupled to an outlet tube to control the release of the oxygen enriched air from accumulator 106 to the user. In an implementation, supply valve 160 is an electromagnetically actuated plunger valve. Supply valve 160 is actuated by controller 400 to control the delivery of oxygen enriched air to a user. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into expansion chamber 162 as depicted in FIG. 1F. In an implementation, expansion chamber 162 may include one or more devices configured to estimate an oxygen concentration of gas passing through the expansion chamber 162. Oxygen enriched air in expansion chamber 162 builds briefly, through release of gas from accumulator 106 by supply valve 160, and then is bled through a small orifice flow restrictor 175 to a flow rate sensor 185 and then to particulate filter 187. Flow restrictor 175 may be a 0.025 D flow restrictor. Other flow restrictor types and sizes may be used. In some implementations, the diameter of the air pathway in the housing may be restricted to create restricted gas flow. Flow rate sensor 185 may be any sensor configured to generate a signal representing the rate of gas flowing through the conduit. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194.

The fluid dynamics of the outlet pathway, coupled with the programmed actuations of supply valve 160, may result in a bolus of oxygen being provided at the correct time and with an amplitude profile that assures rapid delivery into the user's lungs without excessive waste. If the bolus can be delivered in this manner, there may be a linear relationship between the prescribed continuous flow rate and the therapeutically equivalent bolus volume required in pulsed delivery mode for a user at rest with a given breathing pattern. For example, the total volume of the bolus required to emulate continuous-flow prescriptions may be equal to 11 mL for each LPM of prescribed continuous flow rate, i.e., 11 mL for a prescription of 1 LPM; 22 mL for a prescription of 2 LPM; 33 mL for a prescription of 3 LPM; 44 mL for a prescription of 4 LPM; 55 mL for a prescription of 5 LPM; etc. This amount is generally referred to as the LPM equivalent bolus volume. It should be understood that the LPM equivalent may vary between oxygen concentrators due to differences in construction design, tubing size, chamber size, etc. The LPM equivalent will also vary depending on the user's breathing pattern (e.g. breathing rate).

Expansion chamber 162 may include one or more oxygen sensors adapted to determine an oxygen concentration of gas passing through the chamber. In an implementation, the oxygen concentration of gas passing through expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor 165 is an ultrasonic oxygen sensor that includes an ultrasonic emitter 166 and an ultrasonic receiver 168. In some implementations, ultrasonic emitter 166 may include multiple ultrasonic emitters and ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, an ultrasonic sound wave from emitter 166 may be directed through oxygen enriched air disposed in chamber 162 to receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from emitter 166. This phase shift is due to the relatively slow velocity of sound through a gas medium as compared with the relatively fast speed of the electronic pulse through wire. The phase shift, then, is proportional to the distance between the emitter and the receiver and inversely proportional to the speed of sound through the expansion chamber 162. The density of the gas in the chamber affects the speed of sound through the expansion chamber and the density is proportional to the ratio of oxygen to nitrogen in the expansion chamber. Therefore, the phase shift can be used to measure the concentration of oxygen in the expansion chamber. In this manner the relative concentration of oxygen in the accumulator may be estimated as a function of one or more properties of a detected sound wave traveling through the accumulator.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic sensor system may be increased by increasing the distance between the emitter 166 and receiver 168, for example to allow several sound wave cycles to occur between emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of expansion chamber 162 may be reduced or cancelled. The shift caused by a change of the distance between the emitter 166 and receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. patent application Ser. No. 12/163,549, entitled “Oxygen Concentrator Apparatus and Method”, which published as U.S. Publication No. 2009/0065007 A1 on Mar. 12, 2009 and is incorporated herein by reference.

Flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. Flow rate sensor 185 may be coupled to controller 400. The rate of gas flowing through the outlet system may be an indication of the breathing volume of the user. Changes in the flow rate of gas flowing through the outlet system may also be used to determine a breathing rate of the user. Controller 400 may generate a control signal or trigger signal to control actuation of supply valve 160. Such control of actuation of the supply valve may be based on the breathing rate and/or breathing volume of the user, as estimated by flow rate sensor 185.

In some implementations, ultrasonic sensor 165 and, for example, flow rate sensor 185 may provide a measurement of an actual amount of oxygen being provided. For example, flow rate sensor 185 may measure a volume of gas (based on flow rate) provided and ultrasonic sensor 165 may provide the concentration of oxygen of the gas provided. These two measurements together may be used by controller 400 to determine an approximation of the actual amount of oxygen provided to the user.

Oxygen enriched air passes through flow rate sensor 185 to filter 187. Filter 187 removes bacteria, dust, granule particles, etc., prior to providing the oxygen enriched air to the user. The filtered oxygen enriched air passes through filter 187 to connector 190. Connector 190 may be a “Y” connector coupling the outlet of filter 187 to pressure sensor 194 and delivery conduit 192. Pressure sensor 194 may be used to monitor the pressure of the gas passing through conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by either or both of the flow rate sensor 185 and the pressure sensor 194.

Oxygen enriched air may be provided to a user through conduit 192. In an implementation, conduit 192 may be a silicone tube. Conduit 192 may be coupled to a user using an airway delivery device 196, as depicted in FIGS. 1G and 1H. Airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. Airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to oxygen concentrator 100. Mouthpiece 198 may be the only device used to provide oxygen enriched air to the user, or a mouthpiece may be used in combination with a nasal delivery device 196 (e.g., a nasal cannula). As depicted in FIG. 1H, oxygen enriched air may be provided to a user through both a nasal airway delivery device 196 and a mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a drop in pressure proximate to the mouthpiece. Controller 400 of oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, a mouthpiece 198 is used in combination with a nasal airway delivery device 196 (e.g., a nasal cannula) to provide oxygen enriched air to a user, as depicted in FIG. 1H. Both mouthpiece 198 and nasal airway delivery device 196 are coupled to an inhalation sensor. In one implementation, mouthpiece 198 and nasal airway delivery device 196 are coupled to the same inhalation sensor. In an alternate implementation, mouthpiece 198 and nasal airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal airway delivery device 196) proximate to which the onset of inhalation was detected. Alternatively, oxygen enriched air may be provided to both mouthpiece 198 and nasal airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

6. Controller System

Operation of oxygen concentrator 100 may be performed automatically using an internal controller 400 coupled to various components of the oxygen concentrator 100, as described herein. Controller 400 includes one or more processors 410 and internal memory 420, as depicted in FIG. 1B. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, such as the Internet. In the latter instance, the external computing device may provide program instructions to the controller 400 for execution. The term “memory medium” may include two or more memory media that may reside in different locations, e.g., in different computing devices that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 132, 134, 152, 154, 160), oxygen sensor 165, pressure sensor 194, flow rate sensor 185, temperature sensors (not shown), fan 172, and any other component that may be electrically controlled. In some implementations, a separate processor (and/or memory) may be coupled to one or more of the components.

Controller 400 is configured (e.g. programmed by program instructions) to operate oxygen concentrator 100 and is further configured to monitor the oxygen concentrator 100 for malfunction states. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen concentration of the oxygen enriched air passing through expansion chamber 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

Further functions that may be implemented with or by the controller 400 are described in detail in other sections of this disclosure.

7. Control Panel

Control panel 600 serves as an interface between a user and controller 400 to allow the user to initiate predetermined operation modes of the oxygen concentrator 100 and to monitor the status of the system. FIG. 1N depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, dosage buttons 620 to 626, active mode button 630, sleep mode button 635, altitude button 640, and a battery check button 650. In some implementations, one or more of the buttons may have a respective LED that may illuminate when the respective button is pressed, and may power off when the respective button is pressed again. Power button 610 may power the system on or off. If the power button is activated to turn the system off, controller 400 may initiate a shutdown sequence to place the system in a shutdown state (e.g., a state in which both canisters are pressurized). Dosage buttons 620, 622, 624, and 626 allow the prescribed continuous flow rate of oxygen enriched air to be selected (e.g., 1 LPM by button 620, 2 LPM by button 622, 3 LPM by button 624, and 4 LPM by button 626). Altitude button 640 may be activated when a user is going to be in a location at a higher elevation than the oxygen concentrator 100 is regularly used by the user.

Battery check button 650 initiates a battery check routine in the oxygen concentrator 100 which results in a relative battery power remaining LED 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate active mode or sleep mode by pressing button 630 for active mode or button 635 for sleep mode.

8. Pulsed Oxygen Delivery

The main use of an oxygen concentrator 100 is to provide supplemental oxygen to a user. Generally, the continuous flow rate of supplemental oxygen to be provided is prescribed by a physician. Typical prescribed continuous flow rates of supplemental oxygen may range from about 1 LPM to up to about 10 LPM. The most commonly prescribed continuous flow rates are 1 LPM, 2 LPM, 3 LPM, and 4 LPM.

In order to minimize the amount of oxygen enriched air that is needed to be produced to emulate the prescribed continuous flow rate, controller 400 may be programmed to synchronise release of the oxygen enriched air with the user's inhalations, according to a therapy mode known as pulsed oxygen delivery (POD) or demand oxygen delivery. Releasing a bolus of oxygen enriched air to the user as the user inhales may prevent unnecessary oxygen generation (further reducing power requirements) by not releasing oxygen, for example, when the user is exhaling. Reducing the amount of oxygen required may effectively reduce the amount of air compression needed by oxygen concentrator 100 and consequently may reduce the power demand from the compressors.

Oxygen enriched air produced by oxygen concentrator 100 is stored in an oxygen accumulator 106 and, in POD mode, released to the user as the user inhales. The amount of oxygen enriched air provided by the oxygen concentrator 100 is controlled, in part, by supply valve 160. In an implementation, supply valve 160 is opened for a sufficient amount of time to provide the appropriate amount of oxygen enriched air, as estimated by controller 400, to the user. In order to minimize the amount of oxygen required to emulate the prescribed continuous flow rate of a user, the oxygen enriched air may be provided as a bolus soon after the onset of a user's inhalation is detected. For example, the bolus of oxygen enriched air may be provided in the first few milliseconds of a user's inhalation.

In an implementation, pressure sensor 194 may be used to determine the onset of inhalation by the user. For example, the user's inhalation may be detected by using pressure sensor 194. In use, conduit 192 for providing oxygen enriched air is coupled to a user's nose and/or mouth through the nasal airway delivery device 196 and/or mouthpiece 198. The pressure in conduit 192 is therefore representative of the user's airway pressure. At the onset of an inhalation, the user begins to draw air into their body through the nose and/or mouth. As the air is drawn in, a negative pressure is generated at the end of the conduit 192, due, in part, to the venturi action of the air being drawn across the end of the conduit. Controller 400 analyses the pressure signal from the pressure sensor 194 to detect a drop in pressure indicating the onset of inhalation. Upon detection of the onset of inhalation, supply valve 160 is opened to release a bolus of oxygen enriched air from the accumulator 106. A positive change or rise in the pressure indicates an exhalation by the user, upon which the release of oxygen enriched air is discontinued. In one implementation, when a positive pressure change is sensed, supply valve 160 is closed until the next onset of inhalation is detected. Alternatively, supply valve 160 may be closed after a predetermined interval known as the bolus duration. By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through the conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to controller 400 in the oxygen concentrator 100 electronically via a wire or through telemetry such as through Bluetooth™ or other wireless technology.

In some implementations, if the user's current activity level, such as that estimated using the detected user's breathing rate, exceeds a predetermined threshold, controller 400 may implement an alarm (e.g., visual and/or audio) to warn the user that the current breathing rate is exceeding the delivery capacity of the oxygen concentrator 100. For example, the threshold may be set at 40 breaths per minute (BPM).

B. Examples of Vacuum Pressure Swing Adsorption Systems and Methods

1. First Schematic

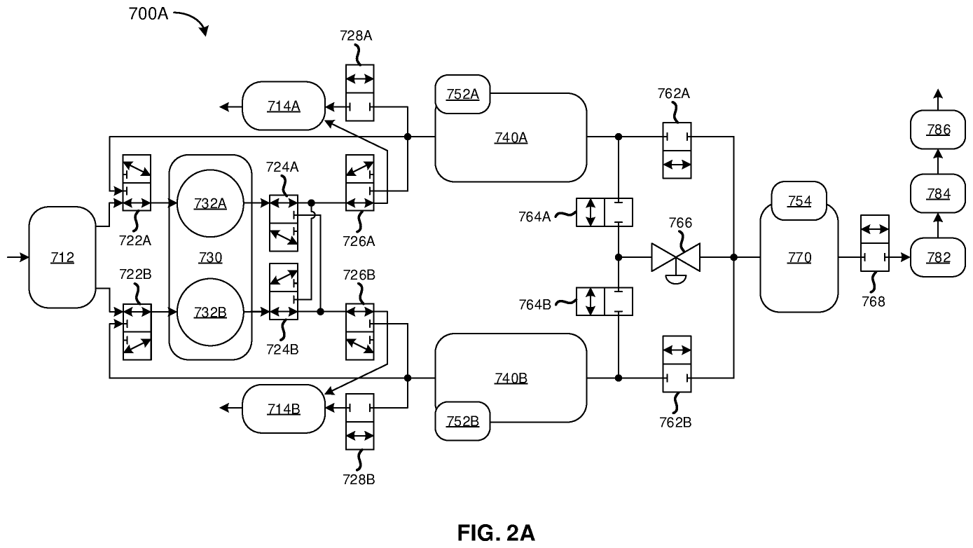

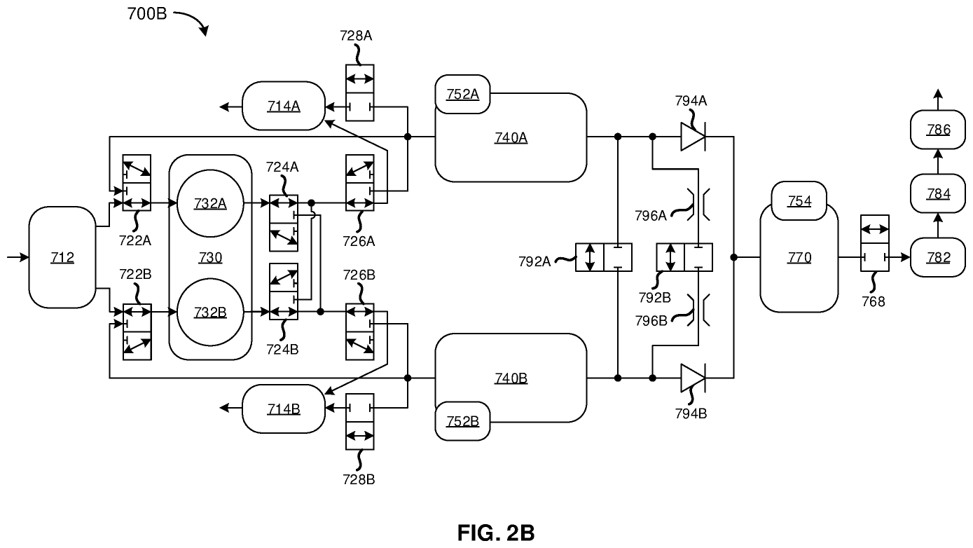

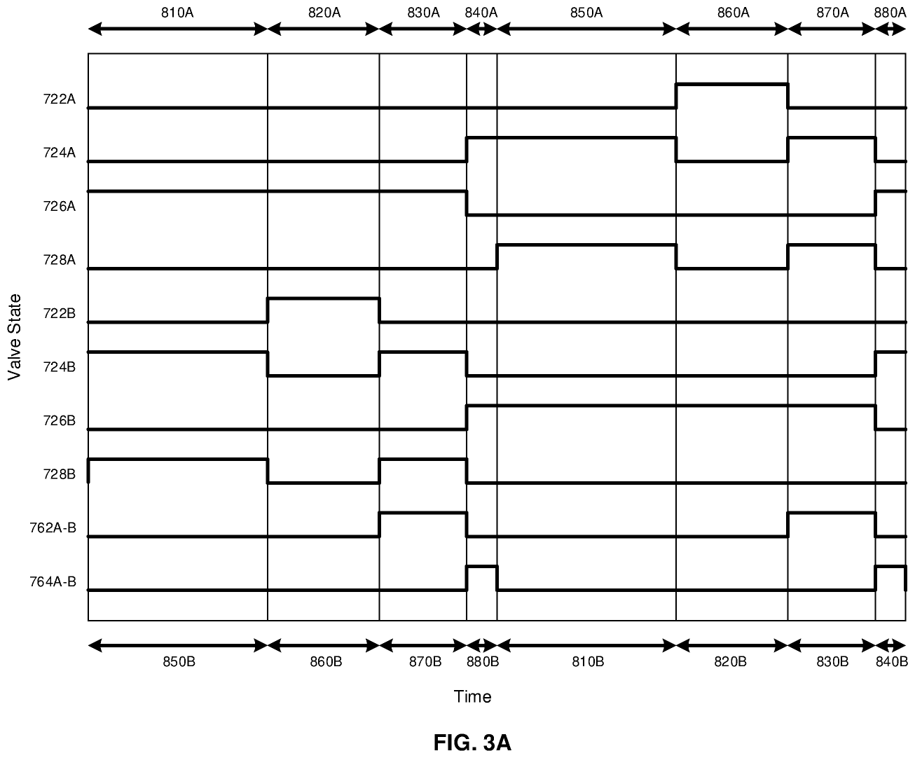

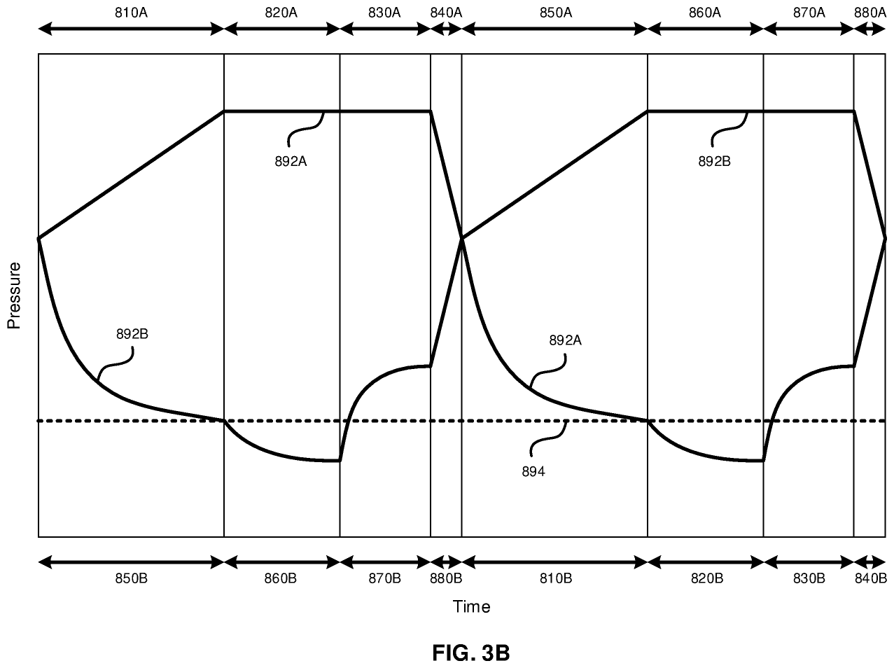

FIGS. 2A, 3A, and 3B illustrate an implementation of an oxygen concentrator 700A. FIG. 2A illustrates a schematic diagram of oxygen concentrator 700A. As described herein, oxygen concentrator 700A uses vacuum pressure swing adsorption (VPSA) processes to produce oxygen enriched air. However, in other implementations, oxygen concentrator 700A may be modified such that it uses purely pressure swing adsorption (PSA) processes or purely vacuum swing adsorption (VSA) processes to produce oxygen enriched air.

Oxygen concentrator 700A may be a portable oxygen concentrator. For example, oxygen concentrator 700A may have a weight and size that allows the oxygen concentrator to be carried by hand and/or in a carrying case. In one implementation, oxygen concentrator 700A has a weight of less than about 20 pounds, less than about 15 pounds, less than about 10 pounds, or less than about 5 pounds. In an implementation, oxygen concentrator 700A has a volume of less than about 1000 cubic inches, less than about 750 cubic inches, less than about 500 cubic inches, less than about 250 cubic inches, or less than about 200 cubic inches.

Oxygen enriched air may be produced from ambient air by pressurizing ambient air in canisters 740A and 740B, which include a gas separation adsorbent. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 2A, ambient air may enter oxygen concentrator 700A through a muffler 712. The ambient air may be drawn into oxygen concentrator 700A by a compressor 730. More specifically, compressor 730 may draw in the ambient air from the surroundings of oxygen concentrator 700A, compress the ambient air, and force the compressed ambient air into one or both canisters 740A and 740B. Muffler 712 may reduce the sound produced by the ambient air as it is drawn into oxygen concentrator 700A by compressor 730. In some implementations, muffler 712 may be a moisture and sound absorbing muffler. For example, a water absorbent or desiccant material (such as a polymer water absorbent material or a zeolite material) may be used to both absorb water from the incoming ambient air and to reduce the sound produced by the ambient air as it is drawn into oxygen concentrator 700A by compressor 730.

Compressor 730 includes pistons 732A and 732B. Due to their ability to displace fluid, a piston and its corresponding cylinder together are referred to herein as a “pump”. Each of the pistons 732A and 732B is configured to draw air into the inlet of its corresponding cylinder as it retracts, compress the air, and force the compressed air out the outlet of the corresponding cylinder as it advances. Depending on whether a vessel is connected to the inlet or the outlet of the cylinder, a piston (and its cylinder) may either pressurize (compress) or depressurize (evacuate) the vessel. When connected via switchable valving, a pump may be configured to selectively compress or evacuate a vessel. Other implementations of pumps, such as rotary (centrifugal) blowers, are contemplated for use in the present technology. A compressor, which comprises at least one pump, may also be referred herein to as a pumping system.

In some implementations, pistons 732A and 732B may reciprocate in antiphase, meaning that during the compressor half-cycle when one piston is advancing in its cylinder, the other piston is retracting. In such implementations, if both pistons are connected to the same vessel, the compressor 730 is said to be carrying out either full-cycle pressurization or full-cycle evacuation of the vessel. If both pistons are connected to different vessels, the compressor 730 is said to be carrying out either half-cycle pressurization or half-cycle evacuation of each vessel.

In some implementations, the ambient air may be pressurized in canisters 740A and 740B to a maximum pressure approximately in a range of 6.5 to 22 pounds per square inch gauge pressure (psig). However, other maximum pressures may also be used, depending on the type of gas separation adsorbent disposed in canisters 740A and 740B. In some implementations, compressor 730 may include additional pistons. Similarly, in some implementations, compressor 730 may be replaced by two or more compressors.

A set of valves (e.g., valves 722A, 724A, 726A, 728A, 722B, 724B, 726B, and 728B) are coupled to compressor 730 and/or canisters 740A and 740B. Using this set of valves, compressor 730 may selectively compress, evacuate, or do both simultaneously. For example, during the VPSA cycle, a first canister, such as canister 740A, may be in a compressed state while a second canister, such as canister 740B, is in an evacuated state. Accordingly, the set of valves may be configured and activated such that pistons 732A and 732B move within the respective cylinders to achieve the compressed state and the evacuated state. In other words, each valve has an ON state and an OFF state, and each valve can be activated (switched between states) to allow, for example, two-piston pressurization of one sieve bed followed by single-piston pressurization and evacuation of both sieve beds to implement portions of a VPSA cycle.

As shown, valves 722A, 724A, 726A, 722B, 724B, and 726B are three-way valves. Furthermore, as shown, valves 728A, 762A, 764A, 728B, 762B, 764B, and 768 are two-way valves. Moreover, valve 766 is a proportional valve through which the flow rate can be controlled, for example by the controller 400. In some implementations, one or more of these valves may be silicon plunger solenoid valves. Plunger valves offer advantages over other kinds of valves by being quiet and having low slippage. However, other types of valves may also be used. In some implementations, a two-step valve actuation voltage may be used to control these valves. For example, a high voltage (e.g., 24 V) may be applied to a valve to open it. The voltage may then be reduced (e.g., to 7 V) to keep the inlet valve open. Using less voltage to keep a valve open may use less power. This reduction in voltage minimizes heat build-up and power consumption to extend run time from the battery. When the power is cut off to the valve, it closes by spring action. In some implementations, the voltage may be applied as a function of time that is not necessarily a stepped response (e.g., a curved downward voltage between an initial 24 V and a final 7 V). In other implementations, different sets of valves may be used to implement the disclosed technology. For example, each of the three-way valves 722A, 724A, 726A, 722B, 724B, and 726B may be replaced by a complementary pair of two-way valves.

In oxygen concentrator 700A, as shown in FIG. 2A, valve 722A selectively connects canister 740A or ambient (e.g., via muffler 712) to an inlet of piston 732A. Valve 724A selectively connects the outlet of piston 732A to either canister 740A (e.g., via valve 726A) or canister 740B (e.g., via valve 726B). Valve 726A selectively connects valve 724A to either canister 740A or ambient (e.g., via muffler 714A). Valve 728A selectively connects canister 740A to ambient (e.g., via muffler 714A). Similarly, valve 722B selectively connects canister 740B or ambient (e.g., via muffler 712) to an inlet of piston 732B. Valve 724B selectively connects the outlet of piston 732B to either canister 740A (e.g., via valve 726A) or canister 740B (e.g., via valve 726B). Valve 726B selectively connects valve 724B to either canister 740B or ambient (e.g., via muffler 714B). Valve 728B selectively connects canister 740B to ambient (e.g., via muffler 714B).

FIG. 3A is an example of a valve activation switch timing diagram (or valve timing diagram) that may be implemented by oxygen concentrator 700A during a VPSA cycle. FIG. 3A illustrates the valve states (ON state or OFF state) of each valve during the VPSA cycle. In particular, FIG. 3A illustrates a relatively lower signal when the power is cut off to a valve, and illustrates a relatively higher signal when a voltage (e.g., 3.3-24 V) is applied to the valve.

FIG. 3B is a graph illustrating corresponding examples of pressure cycles in canisters 740A and 740B during a VPSA cycle. As shown in FIGS. 3A and 3B, stages 810A, 820A, 830A, 840A, 850A, 860A, 870A, and 880A represent various stages of a VPSA cycle performed with canister 740A. Similarly, stages 810B, 820B, 830B, 840B, 850B, 860B, 870B, and 880B represent various stages of a VPSA cycle performed with canister 740B. As shown in FIG. 3B, the pressure cycle in canister 740A is represented by line 892A. Furthermore, the pressure cycle in canister 740B is represented by line 892B. Line 894 represents ambient pressure.

During stage 810A, canister 740A is pressurized by pistons 732A and 732B of compressor 730. As such, the set of valves are configured such that pistons 732A and 732B move within the respective cylinders to pressurize canister 740A. During stage 850B, which is at the same time as stage 810A (e.g., contemporaneous with stage 810A), canister 740B is exhausting nitrogen enriched air. As shown in FIG. 3A, during this time interval, the power is cut off to valves 722A, 724A, 728A, 722B, 726B, 762A, 762B, 764A, and 764B. As such, these valves are in the OFF state. Furthermore, a high voltage (e.g., 3.3-24 V) is applied to valves 726A, 724B, and 728B. As such, these valves are in the ON state and are therefore energized. As a result, ambient air is forced into canister 740A through valves 722A, 722B, 724A, 724B, and 726A. Furthermore, nitrogen enriched air is permitted to flow from canister 740B to the surroundings of oxygen concentrator 700A through valve 728B and muffler 714B. During this time interval, valves 762A, 762B, 764A, and 764B isolate canisters 740A and 740B from accumulator 770 and from each other.

During stage 820A, canister 740A is pressurized by piston 732A of compressor 730. During the contemporaneous stage 860B, canister 740B is evacuated by piston 732B of compressor 730. As shown in FIG. 3A, during this time interval, the power is cut off to valves 722A, 724A, 728A, 724B, 726B, 728B, 762A, 762B, 764A, and 764B. As such, these valves are in the OFF state. Furthermore, a high voltage (e.g., 3.3-24 V) is applied to valves 726A and 722B. As such, these valves are in the ON state and are therefore energized. As a result, ambient air is forced into canister 740A through valves 722A, 724A, and 726A. Furthermore, nitrogen enriched air is drawn out of canister 740B and exhausted into the surroundings of oxygen concentrator 700A through valves 722B, 724B, and 726B and muffler 714B. During this time interval, in VPSA implementations, the pressure in canister 740B falls below ambient pressure. During this time interval, valves 762A, 762B, 764A, and 764B isolate canisters 740A and 740B from accumulator 770 and from each other.

During stage 830A, canister 740A is pressurized by pistons 732A and 732B of compressor 730. During the contemporaneous stage 870B, canister 740B is purged of nitrogen by an oxygen enriched air stream from canister 740A. As shown in FIG. 3A, during this time interval, the power is cut off to valves 722A, 724A, 728A, 722B, 726B, 764A, and 764B. As such, these valves are in the OFF state. Furthermore, a high voltage (e.g., 3.3-24 V) is applied to valves 726A, 724B, 728B, 762A, and 762B. As such, these valves are in the ON state and are therefore energized. As a result, ambient air is forced into canister 740A through valves 722A, 722B, 724A, 724B, and 726A. Furthermore, a portion of the oxygen enriched air in canister 740A is permitted to flow into canister 740B through valves 762A and 762B. Other portions of the oxygen enriched air in canister 740A are permitted to flow into accumulator 770 through valve 762A. As canister 740B is purged by a portion of the oxygen enriched air from canister 740A, nitrogen enriched air is forced out of canister 740B and exhausted into the surroundings of oxygen concentrator 700A through valve 728B and muffler 714B.

During stage 840A and the contemporaneous stage 880B, the pressures of canisters 740A and 740B, respectively, are equalized. Canister 740A is isolated from compressor 730 and mufflers 714A and 714B. Canister 740B is also pressurized by pistons 732A and 732B of compressor 730. As shown in FIG. 3A, during this time interval, the power is cut off to valves 722A, 726A, 728A, 722B, 724B, 728B, 762A, and 762B. As such, these valves are in the OFF state. Furthermore, a high voltage (e.g., 3.3-24 V) is applied to valves 724A, 726B, 764A, and 764B. As such, these valves are in the ON state and are therefore energized. As a result, a portion of the oxygen enriched air in canister 740A is permitted to flow into canister 740B through valves 764A and 764B. Other portions of the oxygen enriched air in canister 740A may be permitted to flow into accumulator 770 through valves 764A and 766. During this time interval, ambient air is also forced into canister 740B through valves 722A, 722B, 724A, 724B, and 726B. At the end of this stage, the pressures in canisters 740A and 740B are approximately equal.

During stage 850A, canister 740A is exhausting nitrogen enriched air. During the contemporaneous stage 810B, canister 740B is pressurized by pistons 732A and 732B of compressor 730. As shown in FIG. 3A, during this time interval, the power is cut off to valves 722A, 726A, 722B, 724B, 728B, 762A, 762B, 764A, and 764B. As such, these valves are in the OFF state. Furthermore, a high voltage (e.g., 3.3-24 V) is applied to valves 724A, 728A, and 726B. As such, these valves are in the ON state and are therefore energized. As a result, nitrogen enriched air is permitted to flow from canister 740A to the surroundings of oxygen concentrator 700A through valve 728A and muffler 714A. Furthermore, ambient air is forced into canister 740B through valves 722A, 722B, 724A, 724B, and 726B. During this time interval, valves 762A, 762B, 764A, and 764B isolate canisters 740A and 740B from accumulator 770 and from each other.

During stage 860A, canister 740A is evacuated by piston 732A of compressor 730. During the contemporaneous stage 820B, canister 740B is pressurized by piston 732B of compressor 730. As shown in FIG. 3A, during this time interval, the power is cut off to valves 724A, 726A, 728A, 722B, 724B, 728B, 762A, 762B, 764A, and 764B. As such, these valves are in the OFF state. Furthermore, a high voltage (e.g., 3.3-24 V) is applied to valves 722A and 726B. As such, these valves are in the ON state and are therefore energized. As a result, nitrogen enriched air is drawn out of canister 740A and exhausted into the surroundings of oxygen concentrator 700A through valves 722A, 724A, and 726A and muffler 714A. Furthermore, ambient air is forced into canister 740B through valves 722B, 724B, and 726B. During this time interval, in VPSA implementations, the pressure in canister 740A falls below ambient pressure. During this time interval, valves 762A, 762B, 764A, and 764B isolate canisters 740A and 740B from accumulator 770 and from each other.

During stage 870A, canister 740A is purged of nitrogen by an oxygen enriched air stream from canister 740B. During the contemporaneous stage 830B, canister 740B is pressurized by pistons 732A and 732B of compressor 730. As shown in FIG. 3A, during this time interval, the power is cut off to valves 722A, 726A, 722B, 724B, 728B, 764A, and 764B. As such, these valves are in the OFF state. Furthermore, a high voltage (e.g., 3.3-24 V) is applied to valves 724A, 728A, 726B, 762A, and 762B. As such, these valves are in the ON state and are therefore energized. As a result, a portion of the oxygen enriched air in canister 740B is permitted to flow into canister 740A through valves 762A and 762B. Other portions of the oxygen enriched air in canister 740B are permitted to flow into accumulator 770 through valve 762B. As canister 740A is purged by a portion of the oxygen enriched air stream from canister 740B, nitrogen enriched air is forced out of canister 740A and exhausted into the surroundings of oxygen concentrator 700A through valve 728A and muffler 714A. Furthermore, during this time interval, ambient air is forced into canister 740B through valves 722A, 722B, 724A, 724B, and 726B.

During stage 880A and the contemporaneous stage 840B, the pressures of canisters 740A and 740B, respectively, are equalized. Canister 740A is also pressurized by pistons 732A and 732B of compressor 730. Canister 740B is isolated from compressor 730 and mufflers 714A and 714B. As shown in FIG. 3A, during this time interval, the power is cut off to valves 722A, 724A, 728A, 722B, 726B, 728B, 762A, and 762B. As such, these valves are in the OFF state. Furthermore, a high voltage (e.g., 3.3-24 V) is applied to valves 726A, 724B, 764A, and 764B. As such, these valves are in the ON state and are therefore energized. As a result, a portion of the oxygen enriched air in canister 740B is permitted to flow into canister 740A through valves 764A and 764B. Other portions of the oxygen enriched air in canister 740B may be permitted to flow into accumulator 770 through valves 764B and 766. During this time interval, ambient air is also forced into canister 740A through valves 722A, 722B, 724A, 724B, and 726A. At the end of these stages, the pressures in canisters 740A and 740B are approximately equal.

Table 1 summarizes the action of each piston and the corresponding state of each canister in the oxygen concentrator 700A over the eight stages of the VPSA cycle implementation illustrated in FIG. 3A.

TABLE 1
Piston actions and canister states over VPSA cycle of FIG. 3A

| | Stage | | | | | | | |
|---|---|---|---|---|---|---|---|---|
| | 810A/ | 820A/ | 830A/ | 840A/ | 850A/ | 860A/ | 870A/ | 880A/ |
| Element | 850B | 860B | 870B | 880B | 810B | 820B | 830B | 840B |
| 732A | P→A | P→A | P→A | P→B | P→B | E←A | P→B | P→A |
| 732B | P→A | E←B | P→A | P→B | P→B | P→B | P→B | P→A |
| 740A | 2P | P | 2P | Vent | Vent | E | Purge | 2P |
| 740B | Vent | E | Purge | 2P | 2P | P | 2P | Vent |

In Table 1, “P→A” means a piston is pressurizing canister 740A; “P→B” means a piston is pressurizing canister 740B; “E←A” means a piston is evacuating canister 740A; “E←B” means a piston is evacuating canister 740B; “P” means a canister is being pressurized by one piston; “2P” means a canister is being pressurized by two pistons; “E” means a canister is being evacuated by one piston; “Purge” means a canister is being purged with a flow of oxygen enriched air from the other canister; and “Vent” means a canister is passively venting nitrogen enriched air to the surroundings of the oxygen concentrator.

As mentioned above, FIG. 3B is a graph illustrating corresponding examples of pressure cycles in canisters 740A and 740B of the oxygen concentrator 700A during a VPSA cycle as implemented using the valve timing illustrated in FIG. 3A. As shown, during most of the VPSA cycle, the pressure of canisters 740A and 740B is above ambient pressure (i.e., line 894). However, in other implementations, oxygen concentrator 700A may operate within a different pressure range. For example, in some implementations, increased portions of the VPSA cycle may be performed at pressures below ambient pressure. As another example, in some implementations, during most of the VPSA cycle, the pressure of canisters 740A and 740B may be below ambient pressure. In such implementations, additional components (e.g., additional valves, flow paths, compressors, etc.) may be used to ensure that a sufficient amount of oxygen enriched gas is collected in accumulator 770 during the VPSA cycle.

As shown in FIG. 2A, pressure sensors 752A, 752B, and 754 may be included in canister 740A, canister 740B, and accumulator 770, respectively. These sensors may be used to measure the pressure of the gas in these components. For example, sensors 752A and 752B may provide pressure information similar to that of lines 892A and 892B, respectively, of FIG. 3B. In some implementations, the durations of stages 810A, 820A, 830A, 840A, 850A, 860A, 870A, 880A, 810B, 820B, 830B, 840B, 850B, 860B, 870B, and/or 880B may be adjusted based on the measured pressures of canister 740A, canister 740B, and/or accumulator 770. In some implementations, sensors 752A and 752B may be used to measure a flow rate between canisters 740A and 740B. Thus, sensors 752A and 752B may be used to balance canisters 740A and 740B in order to maintain the efficiency of oxygen concentrator 700A. In some implementations, additional sensors (e.g., temperature sensors, oxygen sensors, etc.) may be included in canister 740A, canister 740B, and accumulator 770.

The oxygen enriched air stored in accumulator 770 may be delivered to a user through an outlet system comprising supply valve 768, oxygen sensor 782, filter 784, and pressure sensor 786. Supply valve 768 may be used to control the delivery of oxygen enriched air to a user. Oxygen sensor 782 may be used to determine an oxygen concentration of the oxygen enriched air. Filter 784 may be used to filter bacteria, dust, granule particles, etc., prior to delivery of the oxygen enriched air. Pressure sensor 786 may be used to monitor the pressure of the airway of the user.

In some implementations, the outlet system of oxygen concentrator 700A may operate in much the same way as the outlet system of oxygen concentrator 100. For example, supply valve 768, oxygen sensor 782, filter 784, and pressure sensor 786 may operate in much the same way as supply valve 160, oxygen sensor 165, filter 187, and pressure sensor 194, respectively. As another example, in some implementations, the oxygen enriched air may be provided as a bolus soon after the onset of a user's inhalation is detected (e.g., during a POD mode of operation). In some implementations, sensor 786 may be used to detect the onset of a user's inhalation and regulate when a bolus of oxygen enriched air is provided to the user. In some implementations, the outlet system of oxygen concentrator 700A may also include some of the additional components described above in relation to the outlet system of oxygen concentrator 100. For example, oxygen concentrator 700A may include one or more flow restrictors, flow rate sensors, expansion chambers, and/or airway delivery devices.

Additional aspects of oxygen concentrator 100 may also be incorporated into oxygen concentrator 700A. For example, in some implementations, oxygen concentrator 100 may include an outer housing, compression system, canister system, controller system, and/or control panel that are structured and/or configured in much the same way as these components are structured and/or configured in oxygen concentrator 100. Furthermore, in some implementations, some aspects of the separation system of concentrator 100 may also be incorporated into the separation system of oxygen concentrator 700A. For example, one or more check valves may be positioned between canisters 740A and 740B and accumulator 770. As another example, the configuration of the valves between canisters 740A and 740B and accumulator 770 (e.g., valves 762A, 762B, 764A, 764B, and 766) may reconfigured much like the valves and flow restrictors between canisters 302 and 304 and accumulator 106.

The configuration of the valves between canisters 740A and 740B and accumulator 770 may also be reconfigured in other ways. For example, as shown in FIG. 2B, valves 762A, 762B, 764A, 764B, and 766 may be replaced with two-way valves 792A and 792B, check valves 794A and 794B, and flow restrictors 796A and 796B. During operation, valve 792A of oxygen concentrator 700B may be used to equalize the pressures of canisters 740A and 740B (e.g., during stages 840A/880B and 880A/840B). Furthermore, valve 792B may be used to purge canisters 740A and 740B (e.g., during stages 870A/830B and 830A/870B).

In implementations in which the pistons 732A and 732B are in antiphase, during stages 810A and 850B, canister 740A is compressed over the full compressor cycle, alternately by pistons 732A and 732B in each compressor half-cycle, while canister 740B passively exhausts nitrogen enriched air. Thus, the pressure in canister 740A rises more smoothly than if pistons 732A and 732B were in phase. Then during stages 820A and 860B, piston 732B evacuates canister 740B every half cycle while piston 732A compresses canister 740A every other half cycle, so the pressure in 740A rises more slowly than during stages 810A and 850B, and may even plateau as illustrated in FIG. 3B, while the pressure in canister 740B falls below ambient.

The disclosed technology of FIGS. 2A to 3B is more efficient than, for example, conventional VPSA implementations in which a dedicated compressor compresses the canisters and a dedicated vacuum pump evacuates them. As an initial matter, the disclosed technology of FIGS. 2A to 3B uses a single compressor (e.g., compressor 730) to both pressurize and evacuate canisters 740A and 740B. Furthermore, as described above, pistons 732A and 732B are operated in an efficient manner. For example, during stage 810A/850B, canister 740A is rapidly compressed by pistons 732A and 732B, while canister 740B passively exhausts nitrogen enriched air. By taking advantage of the fact that canister 740B will passively exhaust nitrogen enriched air when the pressure of canister 740B is above ambient pressure, pistons 732A and 732B can both be used to rapidly compress canister 740A. Then during stage 830A/870B, canister 740A is compressed by pistons 732A and 732B, while canister 740B is purged of nitrogen enriched air. Then during stage 840A/880B, canister 740B is compressed by pistons 732A and 732B, while canister 740A passively vents into canister 740B. By taking advantage of the fact that canister 740A will passively vent into canister 740B when the pressure of canister 740A is above that of canister 740B, pistons 732A and 732B can both be used to rapidly compress canister 740B to equalize its pressure with that of canister 740A. Similarly advantageous operations are performed during stages 850A/810B, 870A/830B and 880A/840B. These advantages are made possible by the capacity of the disclosed technology to alternate at least one pump (e.g., piston 732A and its cylinder and/or piston 732B and its cylinder) between evacuating a canister and compressing a canister

As explained above, in stage 810A/850B, piston 732B is pressurizing canister 740A. The disclosed technology therefore has a more effective use of piston 732B during stage 810A/850B than conventional VPSA. Similarly, in stage 830A/870B of the disclosed technology, piston 732B is pressurizing canister 740A. The disclosed technology therefore has a more effective use of the second piston during stage 830A/870B than conventional VPSA. Finally, in stage 840A/880B of the disclosed technology, piston 732B is pressurizing canister 740B. The disclosed technology therefore has a more effective use of the second piston during stage 840A/880B than conventional VPSA. Similar advantages may be obtained in stage 850A/810B, stage 870A/830B, and stage 880A/840B. If a component is being put to a more effective use in at least one stage, then a benefit accrues in terms of expected yield for a given mass of components. In other words, using the disclosed technology of FIGS. 2A to 3B, a lighter POC may be able to produce the same output flow rate of oxygen enriched air. This benefit is made possible by the capacity of the disclosed technology of FIGS. 2A to 3B to alternate at least one compressing/evacuating unit (e.g., piston 732A and its cylinder or piston 732B and its cylinder) between evacuating a canister and compressing a canister.

2. Second Schematic

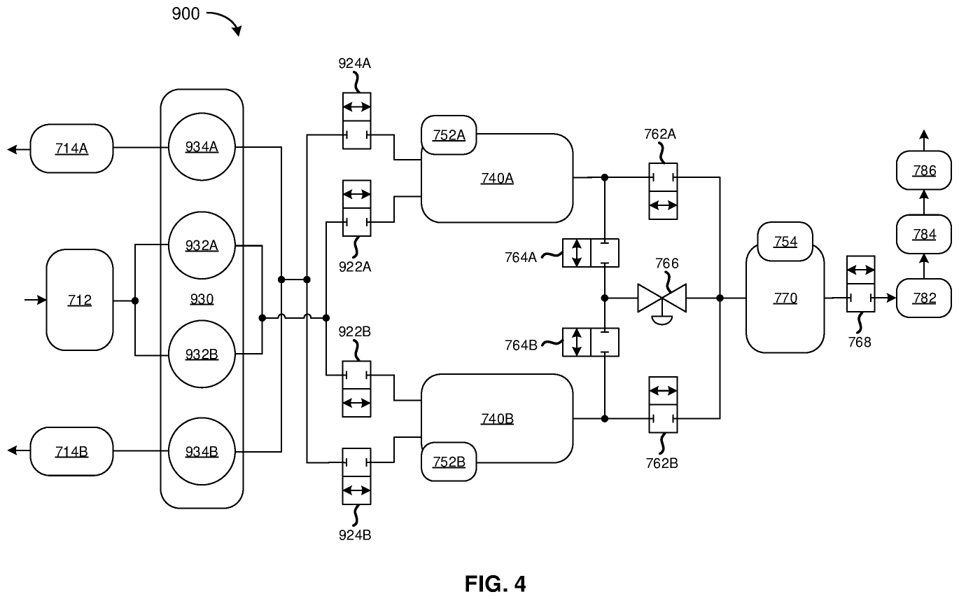

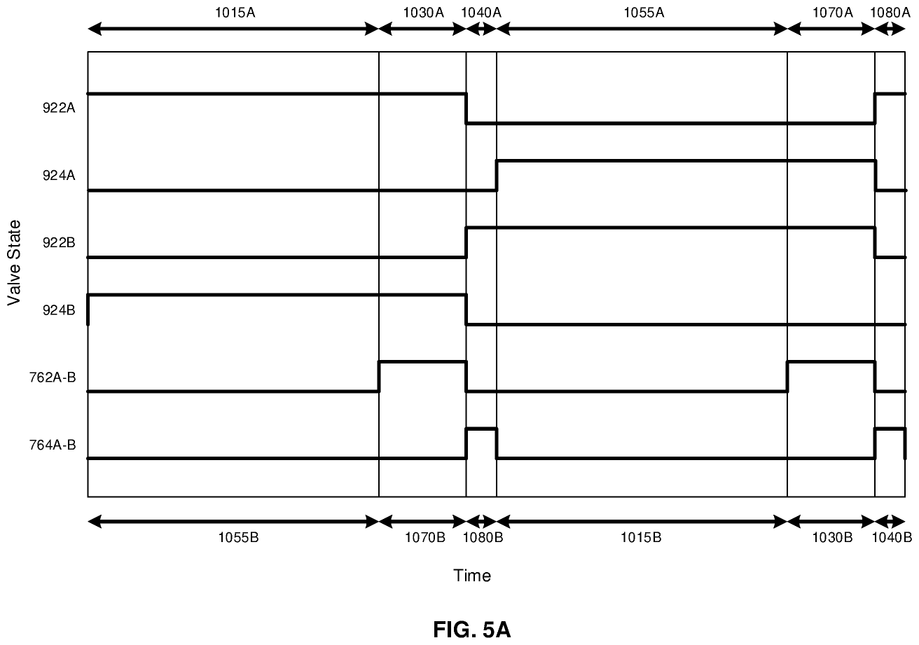

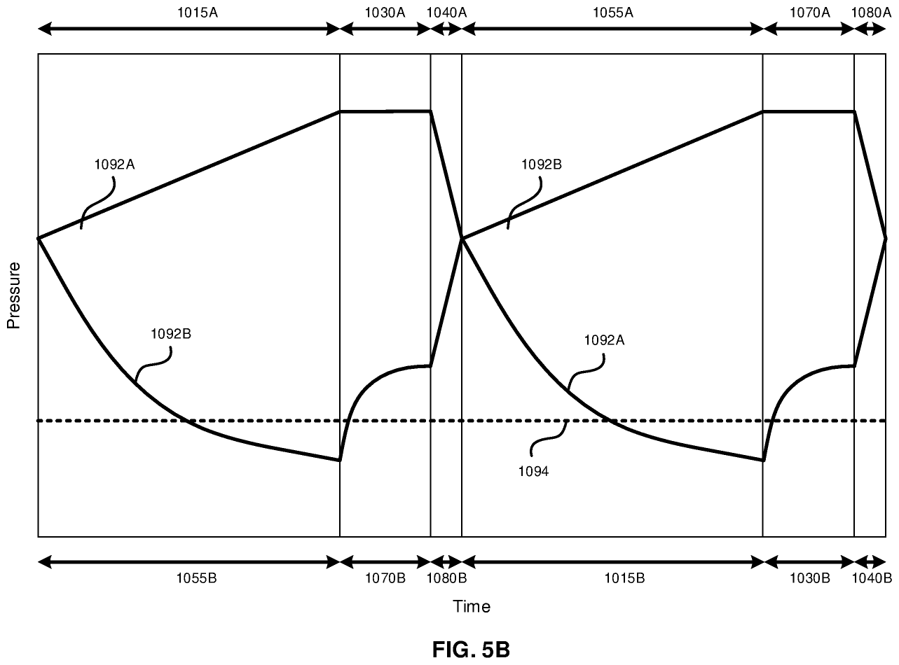

FIGS. 4, 5A, and 5B illustrate an implementation of an oxygen concentrator 900. FIG. 4 illustrates a schematic diagram of oxygen concentrator 900. As described herein, oxygen concentrator 900 uses vacuum pressure swing adsorption (VPSA) processes to produce oxygen enriched air. However, in other implementations, oxygen concentrator 900 may be modified such that it uses purely pressure swing adsorption (PSA) processes or purely vacuum swing adsorption (VSA) processes to produce oxygen enriched air.

As shown, oxygen concentrator 900 includes many of the same components of oxygen concentrator 700A. These components may operate in much the same way that they do in oxygen concentrator 700A. Furthermore, these components may be modified and/or replaced in much the same way. However, in oxygen concentrator 900, valves 722A, 724A, 726A, 728A, 722B, 724B, 726B, and 728B and compressor 730 have been replaced with two-way valves 922A, 924A, 922B, and 924B and compressor 930, respectively.

Compressor 930 includes pistons 932A, 932B, 934A, and 934B. Like pistons 732A and 732B, each of pistons 932A, 932B, 934A, and 934B is configured to draw air into the inlet of its corresponding cylinder as it retracts, compress the air, and force the compressed air out the outlet of the corresponding cylinder as it advances. Depending on whether a vessel is connected to the inlet or the outlet of the cylinder, the piston (and its cylinder) may either pressurize (compress) or depressurize (evacuate) the vessel. Since compressor 930 contains one or more pumps, it may also be referred herein to as a pumping system. In some implementations, pistons 932A and 932B may reciprocate in antiphase, meaning that during the compressor half-cycle when one piston is advancing in its cylinder, the other piston is retracting. Likewise, in some implementations, pistons 934A and 934B may reciprocate in antiphase. Even if pistons 932A and 932B reciprocate in antiphase and pistons 934A and 934B reciprocate in antiphase, there need not be any phase relationship between the reciprocation of pistons 932A and 932B and that of pistons 934A and 934B. In some implementations, the ambient air may be pressurized in canisters 740A and 740B to a pressure approximately in a range of 13-20 pounds per square inch gauge (psig) by compressor 930. However, other pressures may also be used, depending on the type of gas separation adsorbent disposed in canisters 740A and 740B. In some implementations, compressor 930 may include additional pistons. Similarly, in some implementations, compressor 930 may be replaced by two or more compressors. For example, pistons 932A and 932B may be incorporated into one compressor and pistons 934A and 934B may be incorporated into another compressor. Similarly, pistons 932A and 934A may be incorporated into one compressor and pistons 932B and 934B may be incorporated into another compressor.

During operation, pistons 932A and 932B may be configured by a set of valves (e.g., valves 922A, 924A, 922B, and 924B) to pressurize canisters 740A and 740B and pistons 934A and 934B may be configured by the set of valves to evacuate canisters 740A and 740B to implement a VPSA cycle. In contrast, in oxygen concentrator 700A, pistons 732A and 732B may be configured by a set of valves (e.g., valves 722A, 724A, 726A, 728A, 722B, 724B, 726B, and 728B) to alternatively pressurize and evacuate canisters 740A and 740B to implement a VPSA cycle. As a result, fewer valves are required in oxygen concentrator 900. However, in some implementations, additional valves may be incorporated into oxygen concentrator 900.

During a VPSA cycle, oxygen concentrator 900 may cycle through a variety of stages that are similar to stages 810A, 820A, 830A, 840A, 850A, 860A, 870A, 880A, 810B, 820B, 830B, 840B, 850B, 860B, 870B, and/or 880B of FIGS. 3A and 3B. However, due to the configuration of compressor 930, stages 820A, 860A, 820B, and/or 860B may be performed differently. For example, during stage 820A, canister 740A may be pressurized by pistons 932A and 932B (e.g., ambient air may be forced into canister 740A through valve 922A). Furthermore, during the contemporaneous stage 860B, canister 740B may be evacuated by pistons 934A and 934B (e.g., nitrogen enriched air may be drawn out of canister 740B and exhausted into the surroundings of oxygen concentrator 900 through valve 924B).

In some implementations, a VPSA cycle of concentrator 900 may include one or more stages that combine aspects of stages 810A, 820A, 830A, 840A, 850A, 860A, 870A, 880A, 810B, 820B, 830B, 840B, 850B, 860B, 870B, and/or 880B of FIGS. 3A and 3B. FIGS. 5A and 5B illustrate one such example. FIG. 5A is an example of a valve timing diagram that may be implemented by oxygen concentrator 900 during a VPSA cycle. FIG. 5B is a graph illustrating corresponding examples of pressure cycles in canisters 740A and 740B during a VPSA cycle. As shown, stages 1015A, 1030A, 1040A, 1055A, 1070A, and 1080A represent various stages of a VPSA cycle performed with canister 740A. Similarly, stages 1015B, 1030B, 1040B, 1055B, 1070B, and 1080B represent various stages of a VPSA cycle performed with canister 740B. As shown in FIG. 5B, the pressure cycle in canister 740A is represented by line 1092A. Furthermore, the pressure cycle in canister 740B is represented by line 1092B. Line 1094 represents ambient pressure.

During stage 1015A, canister 740A is pressurized by pistons 932A and 932B of compressor 930. During the contemporaneous stage 1055B, canister 740B is evacuated by pistons 934A and 934B of compressor 930 and exhausts nitrogen enriched air. Thus, stage 1015A is comparable to stages 810A and 820A. Similarly, stage 1055B is comparable to stages 850B and 860B. As shown in FIG. 5A, during this time interval, the power is cut off to valves 924A, 922B, 762A, 762B, 764A, and 764B. As such, these valves are in the OFF state. Furthermore, a high voltage (e.g., 3.3-24 V) is applied to valves 922A and 924B. As such, these valves are in the ON state and are therefore energized. As a result, ambient air is forced into canister 740A through valve 922A. Furthermore, nitrogen enriched air is drawn out of canister 740B and exhausted into the surroundings of oxygen concentrator 900 through valve 924B and mufflers 714A and 714B. During this time interval, in VPSA implementations, the pressure in canister 740B falls below ambient pressure while the pressure in canister 740A rises. During this time interval, valves 762A, 762B, 764A, and 764B isolate canisters 740A and 740B from accumulator 770 and from each other.

During stage 1030A, canister 740A is pressurized by pistons 932A and 932B of compressor 930. During the contemporaneous stage 1070B, canister 740B is purged of nitrogen by an oxygen enriched air stream from canister 740A. As shown in FIG. 5A, during this time interval, the power is cut off to valves 924A, 922B, 764A, and 764B. As such, these valves are in the OFF state. Furthermore, a high voltage (e.g., 3.3-24 V) is applied to valves 922A, 924B, 762A, and 762B. As such, these valves are in the ON state and are therefore energized. As a result, ambient air is forced into canister 740A through valve 922A. Furthermore, a portion of the oxygen enriched air in canister 740A is permitted to flow into canister 740B through valves 762A and 762B. Other portions of the oxygen enriched air in canister 740A are permitted to flow into accumulator 770 through valve 762A. As canister 740B is purged of nitrogen by a portion of the oxygen enriched air stream from canister 740A, nitrogen enriched air is drawn and forced out of canister 740B and exhausted into the surroundings of oxygen concentrator 900 through valve 924B and mufflers 714A and 714B. In some implementations, pistons 934A and 934B of compressor 930 may continue to evacuate canister 740B during stage 1070B. In other implementations, pistons 934A and 934B may be idle during stage 1070B. During this time interval, in VPSA implementations, the pressure in canister 740B may rise slightly above ambient pressure, while the pressure in canister 740A may “plateau” at the level reached at the end of stage 1015A.

During stage 1040A and the contemporaneous stage 1080B, the pressures of canisters 740A and 740B, respectively, are equalized. Canister 740B is also pressurized by pistons 932A and 932B of compressor 930. Canister 740A is isolated from compressor 930 and mufflers 714A and 714B. Pistons 934A and 934B are isolated from canisters 740A and 740B and are idle in their respective cylinders. As shown in FIG. 5A, during this time interval, the power is cut off to valves 922A, 924A, 924B, 762A, and 762B. As such, these valves are in the OFF state. Furthermore, a high voltage (e.g., 3.3-24 V) is applied to valves 922B, 764A, and 764B. As a result, a portion of the oxygen enriched air in canister 740A is permitted to vent into canister 740B through valves 764A and 764B. Other portions of the oxygen enriched air in canister 740A may be permitted to flow into accumulator 770 through valves 764A and 766. During this time interval, ambient air is also forced into canister 740B through valve 922B. At the end of these stages, the pressures in canisters 740A and 740B are approximately equal.

During stage 1055A, canister 740A is evacuated by pistons 934A and 934B of compressor 930 and exhausts nitrogen enriched air. During the contemporaneous stage 1015B, canister 740B is pressurized by pistons 932A and 932B of compressor 930. Thus, stage 1055A is comparable to stages 850A and 860A. Similarly, stage 1015B is comparable to stages 810B and 820B. As shown in FIG. 5A, during this time interval, the power is cut off to valves 922A, 924B, 762A, 762B, 764A, and 764B. As such, these valves are in the OFF state. Furthermore, a high voltage (e.g., 3.3-24 V) is applied to valves 924A and 922B. As such, these valves are in the ON state and are therefore energized. As a result, nitrogen enriched air is drawn out of canister 740A and exhausted into the surroundings of oxygen concentrator 900 through valve 924A and mufflers 714A and 714B. Furthermore, ambient air is forced into canister 740B through valve 922B. During this time interval, in VPSA implementations, the pressure in canister 740A falls below ambient pressure while the pressure in canister 740B rises. During this time interval, valves 762A, 762B, 764A, and 764B isolate canisters 740A and 740B from accumulator 770 and from each other.

During stage 1070A, canister 740A is purged of nitrogen by an oxygen enriched air stream from canister 740B. During the contemporaneous stage 1030B, canister 740B is pressurized by pistons 932A and 932B of compressor 930. As shown in FIG. 5A, during this time interval, the power is cut off to valves 922A, 924B, 764A, and 764B. As such, these valves are in the OFF state. Furthermore, a high voltage (e.g., 3.3-24 V) is applied to valves 924A, 922B, 762A, and 762B. As such, these valves are in the ON state and are therefore energized. As a result, a portion of the oxygen enriched air in canister 740B is permitted to flow into canister 740A through valves 762A and 762B. Other portions of the oxygen enriched air in canister 740B are permitted to flow into accumulator 770 through valve 762B. As canister 740A is purged of nitrogen by a portion of the oxygen enriched air stream from canister 740B, nitrogen enriched air is drawn and forced out of canister 740A and exhausted into the surroundings of oxygen concentrator 900 through valve 924A and mufflers 714A and 714B. In some implementations, pistons 934A and 934B of compressor 930 may continue to evacuate canister 740A during stage 1070A. In other implementations, pistons 934A and 934B may be idle during stage 1070A. Furthermore, during this time interval, ambient air is forced into canister 740B through valve 922B. During this time interval, in VPSA implementations, the pressure in canister 740A may rise slightly above ambient pressure, while the pressure in canister 740B may “plateau” at the level reached at the end of stage 1015B.

During stage 1080A and the contemporaneous stage 1040B, the pressures of canisters 740A and 740B, respectively, are equalized. Canister 740A is also pressurized by pistons 932A and 932B of compressor 930. Canister 740B is isolated from compressor 930 and mufflers 714A and 714B. Pistons 934A and 934B are isolated from canisters 740A and 740B and are idle in their respective cylinders. As shown in FIG. 5A, during this time interval, the power is cut off to valves 924A, 922B, 924B, 762A, and 762B. As such, these valves are in the OFF state. Furthermore, a high voltage (e.g., 3.3-24 V) is applied to valves 922A, 764A, and 764B. As such, these valves are in the ON state and are therefore energized. As a result, a portion of the oxygen enriched air in canister 740B is permitted to flow into canister 740A through valves 764A and 764B. Other portions of the oxygen enriched air in canister 740B may be permitted to flow into accumulator 770 through valves 764B and 766. During this time interval, ambient air is also forced into canister 740A through valve 922A. At the end of these stages, the pressures in canisters 740A and 740B are approximately equal.

As mentioned above, FIG. 5B is a graph illustrating corresponding examples of pressure cycles in canisters 740A and 740B during a VPSA cycle. As shown, during most of the VPSA cycle, the pressure of canisters 740A and 740B is above ambient pressure (i.e., line 1094). Furthermore, the overall pressure range in which this VPSA cycle is performed is similar to the overall pressure range in which the VPSA cycle of FIG. 3B is performed. However, in other implementations, oxygen concentrator 900 may operate within a different pressure range. For example, in some implementations, increased portions of the VPSA cycle may be performed at pressures below ambient pressure. As another example, in some implementations, during most of the VPSA cycle, the pressure of canisters 740A and 740B may be below ambient pressure. In such implementations, additional components (e.g., additional valves, flow paths, compressors, etc.) may be used to ensure that a sufficient amount of oxygen enriched gas is collected in accumulator 770 during the VPSA cycle.

In implementations in which pistons 932A and 932B are in antiphase, during stages 1015A and 1055B, canister 740A is compressed over the full compressor cycle, alternately by pistons 932A and 932B in each compressor half-cycle. Thus, the pressure in canister 740A rises more smoothly than if pistons 932A and 932B were in phase or if piston 932B were not present. Likewise, in implementations in which pistons 934A and 934B are in antiphase, during stages 1015A and 1055B, canister 740B is evacuated over the full compressor cycle, alternately by pistons 934A and 934B in each compressor half-cycle. Thus, the pressure in canister 740B falls more smoothly than if pistons 934A and 934B were in phase or if piston 934B were not present. Similarly, during stages 1055A and 1015B, canister 740B is smoothly compressed over the full compressor cycle, alternately by pistons 932A and 932B in each compressor half-cycle, and canister 740A is smoothly evacuated over the full compressor cycle, alternately by pistons 934A and 934B in each compressor half-cycle.

Table 2 summarizes the action of pistons 932A, 932B, 934A, and 934B and the corresponding state of each canister in the oxygen concentrator 900 over the stages of the VPSA cycle implementation illustrated in FIG. 5A using the same notation as in Table 1. Additionally, in Table 2, “2E” means a canister is being evacuated by two pistons and “Idle” means a piston is idle (e.g., not pressurizing or evacuating a canister). The actions of pistons 932B and 934A at each stage are the same as the actions of pistons 932A and 934B, respectively. For ease of comparison with Table 1, stages 1015A/1055B and 1055/1015B have been duplicated in Table 2.

TABLE 2
Piston actions and canister states over VPSA cycle of FIG. 5A

| | Stage | | | | | | | |
|---|---|---|---|---|---|---|---|---|
| | 1015A/ | 1015A/ | 1030A/ | 1040A/ | 1055A/ | 1055A/ | 1070A/ | 1080A/ |
| Element | 1055B | 1055B | 1070B | 1080B | 1015B | 1015B | 1030B | 1040B |
| 932A | P→A | P→A | P→A | P→B | P→B | P→B | P→B | P→A |
| 932B | P→A | P→A | P→A | P→B | P→B | P→B | P→B | P→A |
| 934A | E←B | E←B | E←B | Idle | E←A | E←A | E←A | Idle |
| 934B | E←B | E←B | E←B | Idle | E←A | E←A | E←A | Idle |
| 740A | 2P | 2P | 2P | Vent | 2E | 2E | Purge | 2P |
| 740B | 2E | 2E | Purge | 2P | 2P | 2P | 2P | Vent |

The disclosed technology of FIGS. 4, 5A, and 5B is superior to, for example, conventional VPSA implementations in which a dedicated compressor compresses the canisters and a dedicated vacuum pump evacuates them. As an initial matter, the disclosed technology of FIGS. 4, 5A, and 5B uses a single compressor (e.g., compressor 930) to both pressurize and evacuate canisters 740A and 740B. Furthermore, as described above, pistons 932A, 932B, 934A, and 934B are operated in pairs to efficiently pressurize and evacuate canisters 740A and 740B. For example, in some implementations, each pair of pistons (e.g., pistons 932A and 932B or 934A and 934B) may reciprocate in antiphase. In such implementations, each pair of pistons may carry out either full-cycle pressurization or full-cycle evacuation of canisters 740A and 740B. Thus, the pressure in canisters 740A and 740B rises or falls more smoothly than if, for example, pistons 932B and 934B were not present.

3. Comparison of PSA and VPSA

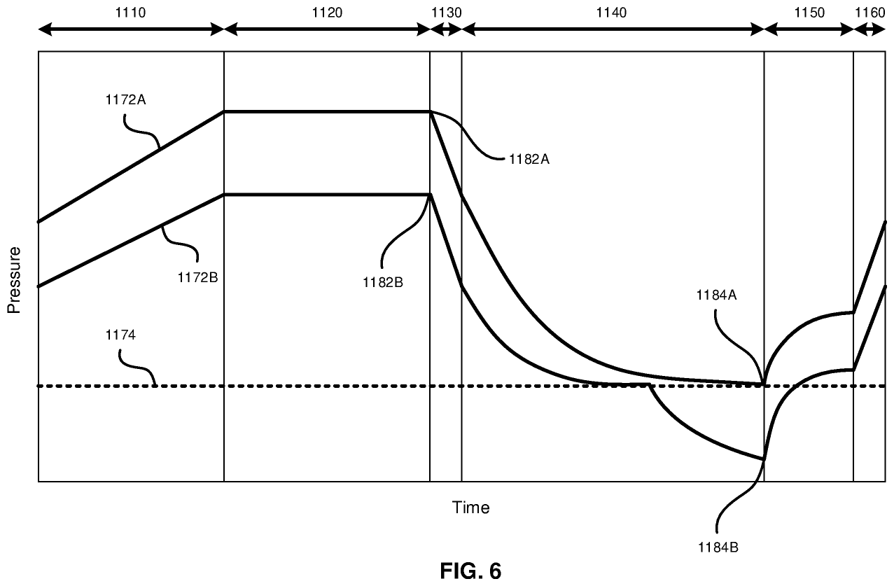

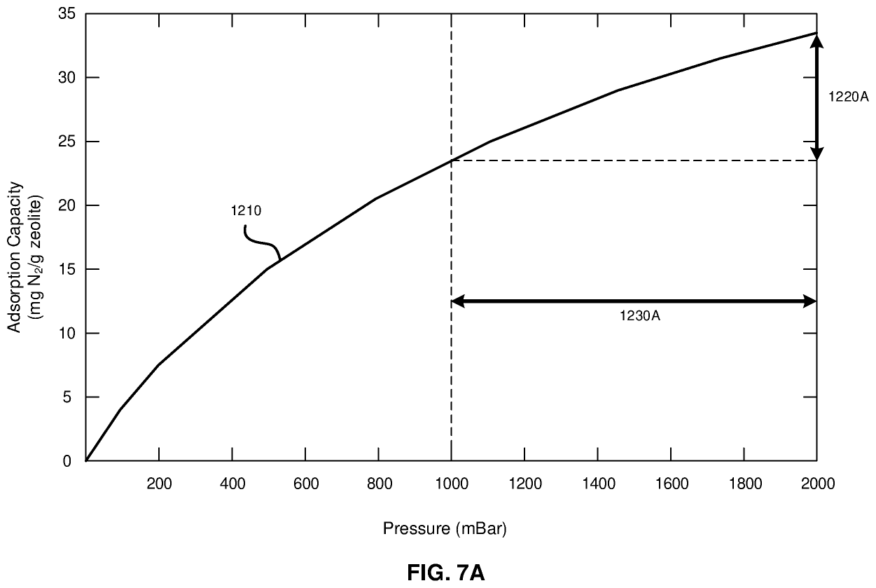

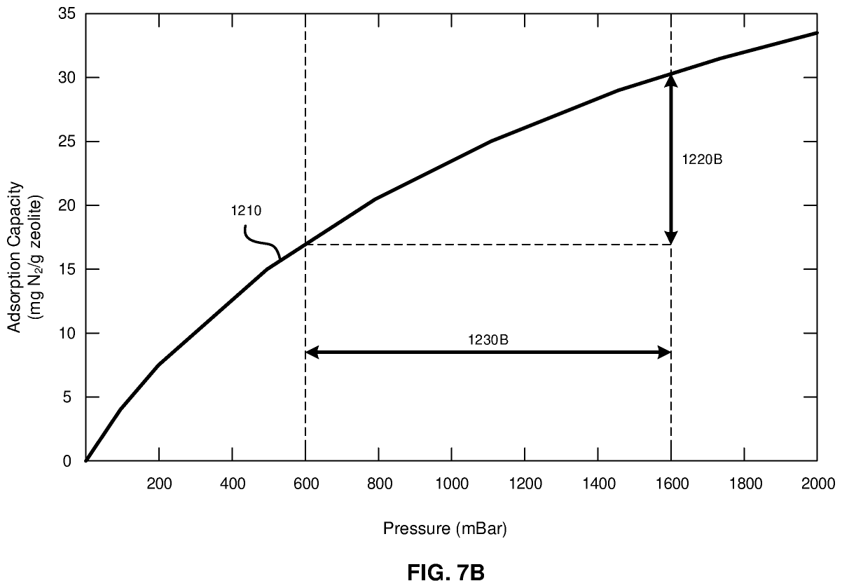

VPSA is a potentially more desirable swing adsorption process than PSA for concentrating oxygen from ambient air. FIGS. 6, 7A, and 7B illustrate some of the differences between pressure swing adsorption (PSA) processes (e.g., as implemented by oxygen concentrator 100) and vacuum pressure swing adsorption (VPSA) processes (e.g., as implemented by oxygen concentrators 700A and 900). For example, in FIG. 6, line 1172A represents the pressure cycle in a canister of an oxygen concentrator during a PSA cycle, and line 1172B represents the pressure cycle in the canister of an oxygen concentrator during a VPSA cycle. Line 1172B may be compared, for example, to line 892A of FIG. 3B. Line 1174 represents ambient pressure (approximately 1000 millibars). During stages 1110 and 1120, the canisters are pressurized and adsorbing nitrogen from an ambient air flow. During stages 1130 and 1160, the canisters are pressure-equalized with one or more other canisters. During stages 1140 and 1150, the canisters are exhausted and purged. Throughout stages 1110, 1120, 1130, 1140, 1150, and 1160, line 1172A fluctuates between a maximum pressure 1182A (e.g., ranging from about 1,200 to 2,000 millibars) and a minimum pressure 1184A (e.g., ambient pressure). Similarly, line 1172B fluctuates between a maximum pressure 1182B (e.g., ranging from about 600 to 1,600 millibars) and a minimum pressure 1184B (e.g., ranging from about 500 to 800 millibars).

Like conventional PSA, VPSA allows the recycling of energy from a de-pressurizing canister to pressurize the other canister prior to bringing the first canister to a partial vacuum state. However, VPSA allows operation at lower average working pressure while maintaining a comparable pressure swing differential to PSA. As shown, the average pressure of the canister represented by line 1172B is lower than the average pressure of the canister represented by line 1172A. Furthermore, line 1172A remains above ambient pressure (i.e., line 1174) throughout the entire PSA cycle, whereas line 1172B drops below ambient pressure (i.e., line 1174) during portions of stages 1140 and 1150. Moreover, maximum pressure 1182A and minimum pressure 1184A of line 1172A are both greater than maximum pressure 1182B and minimum pressure 1184B of line 1172B. As a result, an oxygen concentrator operating in the manner represented by line 1172B may consume less power than an oxygen concentrator operating in the manner represented by line 1172A. Reduced power consumption may be particularly advantageous for portable oxygen concentrators (POCs) operating from one or more batteries with a limited amount of power. Notably, the configurations of oxygen concentrators 700A and 900 further limit the amount of power consumed by using a single compressor to both evacuate and pressurize canisters 740A and 740B. Furthermore, valves 722A, 724A, 726A, 728A, 722B, 724B, 726B, and 728B of oxygen concentrator 700A permit the use of a smaller compressor to generate the same amount of oxygen enriched air. An oxygen concentrator operating in the manner represented by line 1172B may also produce less noise (e.g., from a compressor) than an oxygen concentrator operating in the manner represented by line 1172A. Reduced noise may improve a user's experience and comfort while using the oxygen concentrator. For instance, when an oxygen concentrator is used at night, lower noise levels may lead to better sleep quality or uninterrupted sleep for the user.

In FIGS. 7A and 7B, line 1210 illustrates how the adsorption capacity of a gas separation adsorbent (e.g., zeolite) changes with pressure. As shown, adsorption capacity may increase asymptotically with pressure such that adsorption efficiency decreases at higher pressures. For example, as shown in FIG. 7A, as pressure increases by a value 1230A (e.g., from 1000 millibars to 2000 millibars), the adsorption capacity only increases by a value 1220A (e.g., from 23 mg N₂/g zeolite to 33 mg N₂/g zeolite). In contrast, as shown in FIG. 7B, as pressure increases by a value 1230B (e.g., from 600 millibars to 1600 millibars), which is equal to the pressure increase 1230A, the adsorption capacity increases by a value 1220B (e.g., from 17 mg N₂/g zeolite to 30 mg N₂/g zeolite) that is larger than the increase 1220A. An oxygen concentrator using PSA processes may operate within ranges illustrated in FIG. 7A, whereas an oxygen concentrator using VPSA processes may operate within ranges illustrated in FIG. 7B. Thus, in comparison to an oxygen contractor using PSA processes, an oxygen concentrator using VPSA processes may achieve a higher adsorption rate per unit of pressure differential, as the early part of the adsorbent isotherm has a higher slope at lower pressures and tapers off at higher pressures. This makes VPSA potentially more efficient than PSA in terms of enriched gas yield per unit of power consumed.

In addition to the potential benefits described above, an oxygen concentrator using VPSA processes may regenerate the gas separation adsorbent more efficiently (e.g., during stages 1140 and 1150) and reduce the amount of moisture in the canisters. As explained above, condensation of water inside the canisters of an oxygen concentrator may lead to gradual degradation of the gas separation adsorbents, steadily reducing the ability of the gas separation adsorbents to produce oxygen enriched air. Therefore, by efficiently regenerating the gas separation adsorbent and reducing the amount of moisture in the canisters, the effective lifetime of the gas separation adsorbent may be increased.

C. Label List

| | |
|---|---|
| oxygen concentrator | 100 |
| inlet | 101 |
| inlet | 105 |
| accumulator | 106 |
| muffler | 108 |
| valves | 122 |
| inlet valve | 124 |
| filter | 129 |
| outlet | 130 |
| outlet valve | 132 |
| muffler | 133 |
| outlet valve | 134 |
| spring baffle | 139 |
| check valve | 142 |
| check valve | 144 |
| flow restrictor | 151 |
| valve | 152 |
| flow restrictor | 153 |
| valve | 154 |
| flow restrictor | 155 |
| supply valve | 160 |
| expansion chamber | 162 |
| ultrasonic sensor | 165 |
| emitter | 166 |
| receiver | 168 |
| outer housing | 170 |
| fan | 172 |
| outlet | 173 |
| outlet port | 174 |
| flow restrictor | 175 |
| power supply | 180 |
| flow rate sensor | 185 |
| filter | 187 |
| connector | 190 |
| conduit | 192 |
| pressure sensor | 194 |
| delivery device | 196 |
| mouthpiece | 198 |
| compression system | 200 |
| compressor | 210 |
| compressor outlet | 212 |
| motor | 220 |
| external armature | 230 |
| air transfer device | 240 |
| compressor outlet conduit | 250 |
| canister system | 300 |
| canister | 302 |
| canister | 304 |
| air inlet | 306 |
| housing | 310 |
| base | 315 |
| valve seats | 322 |
| openings | 323 |
| valve seats | 324 |
| outlet | 325 |
| gases | 327 |
| air pathways | 330 |
| valve seats | 332 |
| apertures | 337 |
| conduit | 342 |
| conduit | 344 |
| conduit | 346 |
| opening | 375 |
| controller | 400 |
| processor | 410 |
| memory | 420 |
| housing component | 510 |
| conduit | 530 |
| conduit | 532 |
| conduit | 534 |
| links openings | 542 |
| opening | 544 |
| valve seat | 552 |
| valve seat | 554 |
| control panel | 600 |
| input port | 605 |
| power button | 610 |
| dosage buttons | 620 |
| button | 622 |
| dosage buttons | 624 |
| button | 626 |
| button | 630 |
| mode button | 635 |
| altitude button | 640 |
| battery check button | 650 |
| LED | 655 |
| oxygen concentrator | 700A |
| oxygen concentrator | 700B |
| muffler | 712 |
| muffler | 714A |
| muffler | 714B |
| valves | 722A |
| valves | 722B |
| valves | 724A |
| valves | 724B |
| valves | 726A |
| valves | 726B |
| valve | 728A |
| valves | 728B |
| compressor | 730 |
| piston | 732A |
| piston | 732B |
| canister | 740A |
| canister | 740B |
| sensor | 752A |
| sensor | 752B |
| valve | 762A |
| valve | 762B |
| valve | 764A |
| supply valve | 768 |
| accumulator | 770 |
| oxygen sensor | 782 |
| filter | 784 |
| pressure sensor | 786 |
| valve | 792A |
| valve | 792B |
| check valve | 794A |
| check valve | 794B |
| flow restrictor | 796A |
| flow restrictor | 796B |
| stage | 810A |
| stage | 810B |
| stage | 820A |
| stage | 820B |
| stage | 830A |
| stage | 830B |
| stage | 840A |
| stage | 840B |
| stage | 850A |
| stage | 850B |
| stage | 860A |
| stage | 860B |
| stage | 870A |
| stage | 870B |
| stage | 880A |
| line | 892A |
| line | 892B |
| line | 894 |
| oxygen concentrator | 900 |
| valve | 922A |
| valve | 922B |
| valve | 924A |
| valve | 924B |
| compressor | 930 |
| piston | 932A |
| piston | 932B |
| piston | 934A |
| piston | 934B |
| stage | 1015A |
| stage | 1015B |
| stage | 1030A |
| stage | 1030B |
| stage | 1040A |
| stage | 1040B |
| stage | 1055A |
| stage | 1055B |
| stage | 1070A |
| stage | 1070B |
| stage | 1080A |
| stage | 1080B |
| line | 1092A |
| line | 1092B |
| line | 1094 |
| stage | 1110 |
| stage | 1120 |
| stage | 1130 |
| stage | 1140 |
| stage | 1150 |
| line | 1172A |
| line | 1172B |
| line | 1174 |
| maximum pressure | 1182A |
| maximum pressure | 1182B |
| minimum pressure | 1184A |
| minimum pressure | 1184B |
| line | 1210 |
| value | 1220A |
| value | 1220B |
| value | 1230A |
| value | 1230B |

D. Glossary

For the purposes of the present technology disclosure, in certain forms of the present technology, one or more of the following definitions may apply. In other forms of the present technology, alternative definitions may apply.

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Oxygen enriched air with an oxygen concentration of 80% or greater.

Ambient: In certain forms of the present technology, the term ambient will be taken to mean (i) external of the treatment system or patient, and (ii) immediately surrounding the treatment system or patient.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Patient: A person, whether or not they are suffering from a respiratory disorder.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

E. General Remarks

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

1. An oxygen concentrator for producing oxygen enriched air using vacuum pressure swing adsorption, the oxygen concentrator comprising:
a canister system comprising a first canister for receiving a first gas separation adsorbent, wherein the first gas separation adsorbent is configured to separate at least some nitrogen from a stream of ambient air to produce oxygen enriched air;
a pumping system comprising a first motor-controlled pump;
a set of valves pneumatically coupling the canister system and the pumping system; and
a controller comprising one or more processors, wherein the controller is configured to control operation of the pumping system and the set of valves to:
selectively pneumatically couple the first motor-controlled pump and the first canister so as to pressurize the first canister; and
selectively pneumatically couple the first motor-controlled pump and the first canister so as to evacuate the first canister.

2. The oxygen concentrator of claim 1:
wherein the canister system further comprises a second canister for receiving a second gas separation adsorbent, wherein the second gas separation adsorbent is configured to separate at least some nitrogen from a stream of ambient air to produce oxygen enriched air,
wherein the pumping system further comprises a second motor-controlled pump, and
wherein the controller is further configured to control operation of the pumping system and the set of valves to:
selectively pneumatically couple the second motor-controlled pump and the second canister so as to pressurize the second canister; and
selectively pneumatically couple the second motor-controlled pump and the second canister so as to evacuate the second canister.

3. The oxygen concentrator of claim 2, wherein the controller is further configured to control operation of the pumping system and the set of valves to:
pneumatically couple the first motor-controlled pump and the first canister so as to pressurize the first canister while also selectively pneumatically coupling the second motor-controlled pump and the second canister so as to evacuate the second canister; and
pneumatically couple the first motor-controlled pump and the first canister so as to evacuate the first canister while also selectively pneumatically coupling the second motor-controlled pump and the second canister so as to pressurize the second canister.

4. The oxygen concentrator of claim 3, wherein a pressure of the first canister approaches a first sub-ambient pressure as the first canister is evacuated, and wherein a pressure of the second canister approaches a second sub-ambient pressure as the second canister is evacuated.

5. The oxygen concentrator of claim 4, wherein the first and second sub-ambient pressures range from about 500 to 800 millibars.

6. The oxygen concentrator of any one of claims 2 to 5, wherein the controller is further configured to control operation of the pumping system and the set of valves to:
selectively pneumatically couple the first motor-controlled pump, the second motor-controlled pump, and the first canister so as to pressurize the first canister; and
selectively pneumatically couple the first motor-controlled pump, the second motor-controlled pump, and the second canister so as to pressurize the second canister.

7. The oxygen concentrator of claim 6, wherein the controller is further configured to control operation of the pumping system and the set of valves to:
pneumatically couple the first motor-controlled pump, the second motor-controlled pump, and the first canister so as to pressurize the first canister while also permitting at least a portion of the oxygen enriched air produced by the first canister to purge the second canister; and
pneumatically couple the first motor-controlled pump, the second motor-controlled pump, and the second canister so as to pressurize the second canister while also permitting at least a portion of the oxygen enriched air produced by the second canister to purge the first canister.

8. The oxygen concentrator of claim 6 or 7, wherein the controller is further configured to control operation of the pumping system and the set of valves to:
pneumatically couple the first motor-controlled pump, the second motor-controlled pump, and the first canister so as to pressurize the first canister while also permitting a stream of nitrogen enriched air to be exhausted from the second canister; and
pneumatically couple the first motor-controlled pump, the second motor-controlled pump, and the second canister so as to pressurize the second canister while also permitting a stream of nitrogen enriched air to be exhausted from the first canister.

9. The oxygen concentrator of claim 8, wherein a pressure of the first canister approaches an ambient pressure as the stream of nitrogen enriched air is permitted to be exhausted from the first canister, and wherein a pressure of the second canister approaches the ambient pressure as the stream of nitrogen enriched air is permitted to be exhausted from the second canister.

10. The oxygen concentrator of any one of claims 2 to 9, wherein the controller is configured to control operation of the first and second motor-controlled pumps with a single motor.

11. The oxygen concentrator of any one of claims 2 to 9, wherein the controller is configured to control operation of the first and second motor-controlled pumps with at least two motors.

12. The oxygen concentrator of any one of claims 2 to 11, wherein the first motor-controlled pump comprises a first piston, and wherein the second motor-controlled pump comprises a second piston.

13. The oxygen concentrator of any one of claims 1 to 12, wherein the controller is configured to control operation of the pumping system and the set of valves in a periodic pattern so as to produce oxygen enriched air using vacuum pressure swing adsorption.

14. The oxygen concentrator of any one of claims 1 to 13, wherein the set of valves comprises at least one valve connecting either the first canister or ambient to an inlet of the first motor-controlled pump.

15. The oxygen concentrator of any one of claims 1 to 14, wherein the set of valves comprises a first subset of valves connecting an outlet of the first motor-controlled pump to either the first canister or a second canister.

16. The oxygen concentrator of claim 15, wherein the set of valves comprises a second subset of valves connecting the first subset of valves to the first canister or to ambient.

17. The oxygen concentrator of any one of claims 1 to 16, wherein the set of valves comprises a valve selectively connecting the first canister to ambient.

18. A method for producing oxygen enriched air using vacuum pressure swing adsorption, the method comprising:
selectively pneumatically coupling a first motor-controlled pump of a pumping system and a first canister of a canister system through a set of valves so as to pressurize the first canister, wherein the first canister comprises a first gas separation adsorbent configured to separate at least some nitrogen from a stream of ambient air to produce oxygen enriched air; and
selectively pneumatically coupling the first motor-controlled pump and the first canister through the set of valves so as to evacuate the first canister.

19. The method of claim 18, further comprising:
selectively pneumatically coupling a second motor-controlled pump of the pumping system and a second canister of the canister system through the set of valves so as to pressurize the second canister, wherein the second canister comprises a second gas separation adsorbent configured to separate at least some nitrogen from a stream of ambient air to produce oxygen enriched air; and
selectively pneumatically coupling the second motor-controlled pump and the second canister through the set of valves so as to evacuate the second canister.

20. The method of claim 19:
wherein pneumatically coupling the first motor-controlled pump and the first canister through the set of valves so as to pressurize the first canister is performed while also pneumatically coupling the second motor-controlled pump and the second canister through the set of valves so as to evacuate the second canister, and
wherein pneumatically coupling the first motor-controlled pump and the first canister through the set of valves so as to evacuate the first canister is performed while also pneumatically coupling the second motor-controlled pump and the second canister through the set of valves so as to pressurize the second canister.

21. The method of claim 20, wherein a pressure of the first canister approaches a first sub-ambient pressure as the first canister is evacuated, and wherein a pressure of the second canister approaches a second sub-ambient pressure as the second canister is evacuated.

22. The method of claim 21, wherein the first and second sub-ambient pressures range from about 500 to 800 millibars.

23. The method of any one of claims 19 to 22, further comprising:
selectively pneumatically coupling the first motor-controlled pump, the second motor-controlled pump, and the first canister so as to pressurize the first canister; and
selectively pneumatically coupling the first motor-controlled pump, the second motor-controlled pump, and the second canister so as to pressurize the second canister.

24. The method of claim 23:
wherein pneumatically coupling the first motor-controlled pump, the second motor-controlled pump, and the first canister so as to pressurize the first canister is performed while also permitting at least a portion of oxygen enriched air produced by the first canister to purge the second canister, and
wherein pneumatically coupling the first motor-controlled pump, the second motor-controlled pump, and the second canister so as to pressurize the second canister is performed while also permitting at least a portion of oxygen enriched air produced by the second canister to purge the first canister.

25. The method of claim 23:
wherein pneumatically coupling the first motor-controlled pump, the second motor-controlled pump, and the first canister so as to pressurize the first canister is performed while also permitting a stream of nitrogen enriched air to be exhausted from the second canister, and
wherein pneumatically coupling the first motor-controlled pump, the second motor-controlled pump, and the second canister so as to pressurize the second canister is performed while also permitting a stream of nitrogen enriched air to be exhausted from the first canister.

26. The method of claim 25, wherein a pressure of the first canister approaches an ambient pressure as the stream of nitrogen enriched air is permitted to be exhausted from the first canister, and wherein a pressure of the second canister approaches the ambient pressure as the stream of nitrogen enriched air is permitted to be exhausted from the second canister.

27. An apparatus comprising:
means for receiving a first gas separation adsorbent, wherein the first gas separation adsorbent is configured to separate at least some nitrogen from a stream of ambient air to produce oxygen enriched air;
means for generating compressed air comprising a first motor-controlled pump;
means for pneumatically coupling the means for receiving and the means for generating compressed air; and
means for controlling operation of the means for generating compressed air and the means for pneumatically coupling to:
selectively pneumatically couple the first motor-controlled pump and the means for receiving so as to pressurize the means for receiving; and
selectively pneumatically couple the first motor-controlled pump and the means for receiving so as to evacuate the means for receiving.
